(19) United States
(12) Patent Application Publication
(10) Pub. No.: US 2004/0235061 A1
(43) Pub. Date: Nov. 25, 2004

(54) Methods for selecting and producing animals having a predicted level of immune response, disease resistance or susceptibility, and/or productivity

(76) Inventors: Bruce N. Wilkie, Puslinch (CA); Bonnie A Mallard, Ariss (CA)

Correspondence Address: BERESKIN AND PARR, SCOTIA PLAZA, 40 KING STREET WEST-SUITE 4000 BOX 401, TORONTO, ON, M5H 3Y2, CA

(21) Appl. No.: 10/477,742

(22) PCT Filed: May 24, 2002
(86) PCT No.: PCT/CA02/00733

Related U.S. Application Data
(60) Provisional application No. 60/292,960, filed on May 24, 2001.

(51) Int. Cl. G01N 33/53; G01N 33/567

(52) U.S. Cl. 435/7.2; 119/300

(57) ABSTRACT

The invention relates to methods for selecting animals having a predicted level of immune response, disease resistance or susceptibility, and/or productivity based on an Estimated Breeding Value (EBV) of the animal's immune responsiveness, methods for producing groups of animals having a predicted level of immune response, disease resistance or susceptibility, and/or a predicted productivity based on the EBV; and methods of using such animals.

### FIELD OF THE INVENTION

The invention relates to methods for selecting animals having a predicted level of immune response, disease resistance or susceptibility, and/or productivity based on an Estimated Breeding Value (EBV) of the animal's immune responsiveness; methods for producing groups of animals having a predicted level of immune response, disease resistance or susceptibility, and/or a selected productivity based on the EBV; and methods of using such animals.

### BACKGROUND OF THE INVENTION

The concept of breeding for disease resistance was discussed as early as the 1940's by J. L. Lush (1948) and later by others (Legates and Grinnells 1952; Hutt 1958) as a prophylactic approach to animal health. Original studies focused on identifying resistant livestock during disease outbreaks, recognizing that these animals were often related, and multiplying these groups through within-herd selection (Hutt 1959). This approach gave way to selection based on breed or line differences and established heritability estimates of disease resistance. These methods were successful in specific instances, such as providing reduced mortality from avian leucosis (Hutt and Rasmusen 1982), and improvement of Criolla cattle for heat and tick resistance (de Alba 1978). The principal disadvantages were slow response and high cost. Consequenfly, the consensus among animal breeders was that selection for disease resistance should only be considered when the disease had significant economic impact (Kennedy 1980; McDaniel 1984; Solbu 1984). However, reliance on exogenous methods of disease treatment and/or prevention, such as the use of antibiotics, chemicals, elaborate management schemes, and to some degree vaccination, has caused growing animal and human welfare concerns. Thus contemporary concepts of genetic selection to enhance disease resistance have been met with renewed interest, particularly in light of their potential to reduce the use of chemicals and antibiotics in food producing animals.

Genetic approaches to improved health may prove particularly useful when disease susceptibility is based on a single gene effect (Wood 1981; Horjny 1985; Rothschild et al. 1984; Edfors-Lilja et al. 1995). However, resistance to infectious disease is more often controlled by multiple host resistance genes making selection relatively complex. There is also continued concern that selection for inherent resistance to one disease might be at the expense of susceptibility to other equally important diseases. Furthermore, the agents of disease are genetically complex, and have the ability to express and to vary several virulence factors, necessitating different host response attributes. This information led to the notion of genetic selection for enhanced host resistance as a method to improve broad-based disease resistance, and in the late 1970's attempts to breed laboratory mice for high or low antibody response proved feasible (Biozzi et al. 1979). However, due to negative genetic relationship between the various mechanisms which dictate host immunity, mice with high antibody responses were more resistant to extracellular pathogens, but had increased susceptibility to intracellular pathogens, such as Salnonella typhimurium, which are better controlled by enhanced phagocytic cell function and cell mediated immunity (CMI)(Biozzi et al. 1979). Low line mice demonstrated the reverse features of host resistance. Meanwhile, in swine and other livestock, expanding knowledge regarding the phenotypic and genotypic variation of host resistance mechanisms, and the genes which influence these responses, contributed to a rationale for genetic selection based on aspects of immune response (Gavora and Spencer 1983; Buschmann et al. 1985; Mallard et al. 1989).

In a variety of species, including pigs, genes of the Major Histocompatibility Complex (TMC) were reported to control approximately ten percent of the variation in immune response parameters and to have relevance to the outcome of infection (Biozzi et al. 1979; Mallard et al. 1989). Indirect selection for improved resistance to Marek's disease was initially applied to commercial chickens based on expression of particular MHC genes with no adverse effects on production (Simonsen 1987). In fact, a synergism was reported between genetic resistance to Marek's disease and response to vaccination (Gavora and Spencer 1983). However, the disadvantage remained that the MFC is only one set of many groups of genes mediating host resistance, and with the possible appearance of more virulent pathogenic strains it may prove necessary to modify the selection criteria. Furthermore, this type of selection could result in the loss of valuable genes required to combat the ever changing set of pathogens.

Wilkie et al. devised a multi-trait selection index using EBVs of at least four immune response traits as a basis to improve broad-based disease resistance (PCT Application No. CA93/00533, published as WO 94/14064). The procedure for determining an EBV involved determining the animal's heritable humoral immunity traits by testing an animal's response to at least two tests one of which is a general measure and the other antigen specific; and determining heritable cell-mediated immunity traits by testing the animal's response to at least two tests one of which is a general measure and the other antigen specific.

### SUMMARY OF THE INVENTION

The present inventors have developed an improved method for identifying animals with a predicted immune response, disease resistance or susceptibility, and/or productivity. The method uses Estimated Breeding Values (EBV) of two specific immune response traits that are highly heritable and thus are passed on from one generation to the next. The method is more efficient and less costly than prior art methods in that it requires only two specific determinations to establish an EBV. The genetic gain increases in the shorter period since only two determinations are made.

Broadly stated, the present invention relates to a method for predicting an animal's level of immune response, disease resistance or susceptibility, and/or productivity, based on an EBV of the animal's immune responsiveness, comprising:

(i) determining a heritable antibody response trait of a test animal by measuring in the test animal the levels of antibody which are specifically induced to a predetermined antigen;

(ii) determining a cell-mediated immune response trait of the test animal by measuring in the test animal a cell-mediated immune response which is specifically induced to a predetermined antigen;

(iii) calculating an EBV for the test animal which is based on the determinations in (i) and (ii); and

(iv) comparing the test animal's EBV to EBVs for other animals within a population of animals, and thereby assigning the test animal to a high, low, or control EBV group, wherein a high, low, or control EBV correlates with a predicted level of immune response, disease resistance or susceptibility, and/or productivity in the test animal.

The invention also relates to a method for obtaining a group of animals which has a predicted level of immune response, disease resistance or susceptibility, and/or a group of animals which has a predicted productivity which comprises:

(i) determining a heritable antibody response trait of a test animal by measuring in the test animal the levels of antibody which are specifically induced to a predetermined antigen;

(ii) determining a CMIR trait of the test animal by measuring in the test animal a CMIR which is specifically induced to a predetermined antigen;

(iii) calculating an EBV for the test animal based on the determinations in (i) and (ii); and

(iv) comparing the test animal's EBV to EBVs obtained for other animals within a population of animals and thereby assigning the test animal to a high, low or control EBV group; and

(v) selecting animals in one of the high, low or control EBV groups and breeding the animals to produce a group of animals which have a predicted level of immune response, disease resistance or susceptibility, and/or a group of animals which has a predicted productivity.

The invention further relates to a method of determining the efficacy of a vaccine, drug or other treatment in an animal comprising:

(i) determining a heritable antibody response trait of a test animal by measuring in the test animal the levels of antibody which are specifically induced to a predetermined antigen;

(ii) determining a cell-mediated immune response trait of the test animal by measuring in the test animal a cell-mediated immune response which is specifically induced to a predetermined antigen;

(iii) calculating an EBV for the test animal based on the determinations in (i) and (ii);

(iv) comparing the test animal's EBV to EBVs obtained for other animals within a population of animals and thereby assigning the test animal to a high, low or control EBV group; and

(v) administering the vaccine, drug, or other treatment to animals in one or more of the high, low or control EBV groups, and comparing the responses to the vaccine, drug or other treatment in one or more of the high, low and control EBV groups, wherein a positive response to the vaccine, drug or other treatment in the high EBV group only, indicates that the vaccine, drug or other treatment has low efficacy, and wherein a positive response to the vaccine, drug or other treatment in the high, control and low EBV groups indicates that the vaccine, drug or other treatment has high efficacy.

In one embodiment, the invention provides a method for predicting the level of immune response, disease resistance or susceptibility, and/or productivity of a test animal within a population of animals based on an EBV of the animal's immune responsiveness comprising:

(a) immunizing the test animal at least once with at least one antigen which can evoke a specific antibody response;

(b) for the test animal, measuring a specific antibody response to the at least one antigen at least once;

(c) exposing the test animal to an antigen which can evoke a specific CMIR; and

(d) measuring at least one indicator of the CMIR of the test animal, (e) calculating the EBV for the test animal based on the determinations in (b) and (d); and

(f) comparing the test animal's EBV to EBVs obtained for the other animals within the population of animals and thereby assigning the test animal to a high, low, or control EBV group, wherein a high, low or control EBV correlates with a predicted level of immune response, disease resistance or susceptibility, and/or productivity in the test animal.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### DETAILED DESCRIPTION OF THE INVENTION

Definitions

“Disease resistance or susceptibility” refers to resistance or susceptibility to clinical or subclinical conditions of several potential aetiologies including infectious, neoplastic, or stress-related. Examples of diseases resulting from infectious agents include but are not limited to peritonitis, pleuritis, pericarditis, mastitis, dermititis, enteritis, pneumonia, encephalitis, myelitis, and metritis. The term “disease resistance or susceptibility” herein also refers to responsiveness to vaccination and to therapy such as antibiotics.

“Productivity” as used herein, refers to the rate of growth of an animal including the time to reach a selected market weight, feed conversion efficiency, and reproductive performance including the number of live animals/litter, and the number of undeformed animals per litter.

“Animal” as used herein includes all members of the animal kingdom. The methods of the present invention may be applied to a wide variety of species. Preferably, they are applied to commercially important animal species including: swine; cattle; sheep; avian species, such as chickens, and fish; horses; dogs; and cats.

“Antigen” as used herein, refers to any agent to which an animal is exposed and elicits the specified immune response. Suitable antigens for use in the present invention can be of animal, bacterial, viral, synthetic, or other origin. In choosing suitable antigens for the present invention, the antigens are preferably ones to which the animal is not normally exposed, and preferably one to which they have not been exposed. A person skilled in the art would appreciate that the preferred antigens will depend on the animal species used.

“Estimated Breeding Value” or “EBV” as used herein, refers to a determined numeric value of a phenotypic trait which takes into account measurements of the trait in the individual and its relatives, thereby predicting the genetic ability of the individual to transmit the trait to its offspring.

“Population” as used herein refers to a group of animals of the same species in which the measurements are obtained. Population as used herein can also refer to a sample of the population, in so far as obtaining the EBV levels in a significant sample of a population can enable one to estimate or predict the EBV values of other related animals within the population.

“Stress” as defined herein, is any acute or chronic increase in physical, metabolic, or production-related pressure to the animal. It is the sum of the biological reactions to any adverse stimulus, physical, metabolic, mental or emotional, internal or external, that tends to disturb an organisms homeostasis.

The methods of the invention may be used to select animals having a predicted level of immune response, disease resistance or susceptibility, and/or a predicted productivity; to obtain a group of animals which has a predicted level of immune response, disease resistance or susceptibility, and/or a predicted productivity; and to determine the efficacy of a vaccine, drug or other treatment in an animal.

Antibody Response

The methods of the present invention involve determining a heritable antibody response trait of an animal by measuring in the animal the levels of antibody which are specific to a predetermined antigen. Preferred antigens which may be used to assess antibody response include soluble antigens, and antigens that are poor immunogens. Examples of antigens which may be used in the methods of the invention include Hen Egg White Lysozyme (HEWL), or similar antigens such as, ovalbumin, sheep red blood cells, and synthetic peptides such as tyrosine, glycine, alanine copolymer ((TG)-A-L). Immunization may also be by administration of nudeic acids specific for the immunizing agents or its components. A person skilled in the art would understand that there are many types of antigens and methods to induce an antibody response. The invention extends to cover all such antigens and methods.

A standard protocol for immunization may be used for assessing antibody response. For example, the antigen may be introduced into the animal through intraperitoneal, intramuscular, intraocular, or subcutaneous injections, in conjunction with an adjuvant such as Quil-A and Freund's Complete Adjuvant. Following a primary immunization and, preferably, one secondary immunization, samples of serum are collected at appropriate times and antibodies are measured. A wide variety of assays may be utilized to measure the antibodies which are reactive against the predetermined antigen, including for example enzyme-linked immuno-sorbent assays (ELISA), countercurrent immuno-electrophoresis, radioimmunoassays, radioimmunoprecipitations, haemogglutination and passive haemogluttination, dot blot assays, inhibition or competition assays, and sandwich assays (see U.S. Pat. Nos. 4,376,110 and 4,186,530; see also Antibodies: A Laboratory Manual, Harlow and Lane (eds.), Cold Spring Harbor Laboratory Press, 1988).

Cell-Mediated Immune Response

The method also involves determining a CMIR trait of an animal by measuring in the animal a cell-mediated immune response which is specific to a predetermined antigen. Suitable indicators of CMIR which can be used to measure CMIR in an animal include, but are not limited to, the measurement of one or more predetermined cytokines [for example, as described in L. T. Jordan et al. “Interferon Induction in SLA-Defined Pigs”, Res. Vet. Sci. 58:282-283, 1995; N. R. Jayagopala Reddy et al., “Construction Of An Internal Control To Quantitate Multiple Porcine Cytokine mRNAs by rtPCR”, BioTechniques 21:868-875, 1996; N. R. Reddy, B. N. Wilkie, “Quantitation of Porcine Cytokine and beta-2-Immunoglobulin in RNA Expression by Reverse Transcription Polymerase Chain Reaction”, J. Immunol. Methods 233:83-93 (2000); W. C. Brown et al., “Bovine Type 1 And Type 2 Responses”, Vet. Immunl. Immunopath 63:45-55, 1998]; measuring delayed-type hypersensitivity (DTHI) (for example as described in Mallard, 1992, PCT/CA93/00533); and measuring in vitro lymphocyte proliferation to at least one antigen (for example, as described in Mallard B. A. et al., Animal Biotech 1992, 3(2):257-280).

Further, CMIR may be assessed by measuring delayed-type hypersensitivity (DTH) induced by a live agent such as Bacillus Calmette Guérin (BCG), or an inactive agent such as killed Mycobacterium or a derivative thereof, such as a purified protein derived (PPD) from a strain of. Mycobacterium. The CMIR may also be assessed by measuring contact sensitivity. Standard protocols may be used to induce CMIR and conventional cellular assays, such as cell-mediated cytotoxicity, antigen-induced blastogenesis, cytokine assays, measurement of cell surface markers such as CD4, CD5 or CD8, or combinations thereof, may be used to measure the response. For example, pigs may receive BCG intradermally and subsequently PPD intradermally, and the cutaneous responses, i.e. DTH may be measured by double skin fold thickness. Further, cytokines, for example, interleukin-2 (IL-2) and interferon-g (IFN-g) may also be measured in vitro or in vivo using conventional methods. A person skilled in the art would understand that there are many methods to induce and assess a CMIR. The invention extends to all such methods.

In a preferred embodiment of the invention, the predetermined antigen which specifically induces an antibody response and the predetermined antigen which specifically induces a CMIR are different antigens. Further, the antigens are preferably selected from a group of antigens to which the animals are not normally exposed and most preferably have not been previously exposed.

Estimated Breeding Values of Immune Response

In an embodiment of the invention the heritable antibody response trait of a test animal is determined by:

(a) immunizing the test animal at least once with at least one antigen which can evoke a specific antibody response;

(b) for the test animal, measuring a specific antibody response to the at least one antigen at least once;

In another embodiment of the invention, the CMIR trait of the animal is determined by:

(c) exposing the test animal to an antigen which can evoke a specific CMIR; and

(d) measuring at least one indicator of the CMIR of the test animal. Preferably the test animal is immunized at least two times with with at least one antigen which can evoke a specific antibody response and is exposed at least two times to an antigen which can evoke a specific CMIR.-,

The antibody and CMIRs may be assessed at a time in the animal's life when they are stressed, and/or at most risk for disease, and/or at a time that ensures the least amount of interference with accurate measurement of the immune responses. For example, to practice the method of the invention to identify high immune response or low immune response pigs with a predicted level of immune response, disease resistance or susceptibility, and/or productivity, the pigs may be immunized beginning at a time when interfering maternal antibodies are minimal, particularly to inert antigens not previously encountered; for example, after weaning which is typically at an average age of 21 days. For ranking dairy cows for resistance to mastitis, immunization may occur in the pre- and postpartum periods. The two immune traits may also be continuously assessed. It will also be appreciated that the animals may be pre-screened and selected using other phenotypic indices prior to determirnig the two immune response traits described herein.

The method of the invention also involves calculating the EBV for an animal based on the animal's specific antibody and cell-mediated immune responsiveness. As stated above, “Estimated Breeding Value” or “EBV” as used herein, refers to a determined numeric value of a phenotypic trait which takes into account measurements of the trait in the individual and its relatives, thereby predicting the genetic ability of the individual to transmit the trait to its offspring. Generally, the observations on the antibody and CMIR-traits are ranked using normal scores. Estimates of heritabilities of the standardized records are then obtained by a restricted maximum likelihood model, and the solutions from the restricted maximum likelihood analyses are used to compute an EBV for each of the two immune response traits for an animal. The EBVs are combined for the two traits to provide a total EBV for an animal. The animals are ranked according to total EBV and assigned to high, control, or low breeding groups.

Animals may be assigned to a particular group i.e. high, control, or low groups, based on their total EBVs. The EBV ranking of an animal depends on where it fits on a continuum established amongst all tested animals. For instance, animals having an EBV within a top percentage of the continuum may be assigned to the high group. Animals having an EBV within a bottom percentage of the continuum may be assigned to the low group. Animals having an EBV between the high and low groups may be assigned to the control group. The control EBV group is a random bred population used for comparison. This control group permits random drift of EBV within a species to be taken into account when ranking the EBV of an animal. Therefore, selected groups are provided that exhibit specific immune response, disease resistance or susceptibility, and/or productivity. Typically, the animals assigned to the high group differ from the animals assigned to the low group, or other non-selected animals within the population, in that they have (a) a greater ability to resist disease, and pass such resistance to offspring, (b) greater productivity, (c) a greater ability to respond to vaccination, and/or (d) they produce antibodies of higher binding strengths (avidity) in response to an immunogen indicating a superior immune response. Animals in one of the high, low or control EBV groups can be selected for breeding to produce a group of animals which have a predicted level of immune response, disease resistance or susceptibility, and/or a group of animals which has a predicted productivity. For example, animals in a high EBV group may be bred to produce a group of animals which have a high resistance to disease, or high productivity or high response to vaccines. Groups of animals may also be produced that have very low resistance to disease or response to vaccines. Traditional hereditary breeding techniques can be used (Veterinary Genetics, F. W. Nicholas, Oxford Science Publications, 1987; D. S. Falconer. An introduction to quantitative genetics. Longman, London, 1981). A person skilled in the art upon reading the present description would appreciate that the methods of the invention can also be used to predict the EBV of an animal if one has knowledge of the EBV ranking of at least one of the animal's relatives. Factors which would increase the accuracy of the prediction of such an EBV ranking of an animal, include but are not limited to:

(i) degree of separation from the animal (the knowledge of the ranking of the animal's full siblings and parents would result in a better prediction than with knowledge of the ranking of only cousins or partial siblings);

(ii) the amount of data (the greater the database of knowledge of the EBVs of one's relatives, the better the prediction); and

(iii) the similarity of environmental factors.

The EBVs for the two immnune response traits are combined with equal weighting to derive an immune response index (IR). EBVs for production traits, for example, backfat and growth, are used to derive a production index (PI), which may be combined with IR to derive a selection index (SI). IR and PI may be weighted variably to give emphasis to immune response or production traits. The methods of the invention may be used to establish specific selection indices for different animal species and different breeds.

Efficacy of Vaccines, Drugs and Other Treatments

The animals having predicted immune response, disease resistance or susceptibility, and/or response to vaccines can be used in vaccine development and screening programs and to determine the efficacy of new drugs, vaccines and other treatments. In particular, the efficacy of a vaccine, drug or other treatment in an animal can be determined by administering the vaccine, drug or other treatment to animals in one or more of the high, low or control EBV groups, and comparing the responses to the vaccine, drug or other treatment in one or more of the low, high and control EBV groups to determine the efficacy of the vaccine, drug or other treatment. The theory being that if the drug or vaccine works on animals with low EBVs, it should work on animals with higher EBVs. “Drug” as used herein covers all therapeutic and prophylactic treatments.

More particularly the method of determining the efficacy of a vaccine, drug or other treatment in an animal in accordance with the present invention preferably comprises:

(i) determining a heritable antibody response trait of a test animal by measuring in the test animal the levels of antibody which are specifically induced to a predetermined antigen;

(ii) determining a CMIR trait of the test animal by measuring in the test animal a cell-mediated immune response which is specifically induced to a predetermined antigen;

(iii) calculating an EBV for the test animal based on the determinations in (i) and (ii);

(iv) comparing the test animal's EBV to EBVs obtained for other animals within a population of animals and thereby assigning the test animal to a high, low or control EBV group; and

(v) administering the vaccine, drug, or other treatment to animals in one or more of the high, low or control EBV groups, and comparing the responses to the vaccine, drug or other treatment in one or more of the high, low and control EBV groups, wherein a positive response to the vaccine, drug or other treatment in the high EBV group only, indicates that the vaccine, drug or other treatment has low efficacy, and wherein a positive response to the vaccine, drug or other treatment in the high, control and low EBV groups indicates that the vaccine, drug or other treatment has high efficacy.

The method of the invention may also be used to study and determine the virulence traits, or the means whereby disease-producing microorganisms produce disease, in susceptible individuals.

Stress

The methods of the present invention can also be used to select for animals and/or develop a group of animals with predicted levels of immune response, disease resistance or susceptibility, and/or productivity during stress. An association between stress and disease resistance is known (T. Molitor and L. Schwandtdt, “Role Of Stress On Mediating Disease In Animals”, Proc. Stress Symposia: Mechanisms, Responses, Management. Ed., N. H. Granholm, South Dakota State University Press, Apr. 6-7, 1993). Further it has been suggested that stress can lead to a compromised immune system (T. Molitor and L. Schwandtdt, “Role Of Stress On Mediating Disease In Animals”, Proc. Stress Symposia: Mechanisms, Responses, Management. Ed., N. H. Granholm, South Dakaota State University Press, Apr. 6-7, 1993/Morrow-Tesch J. L. et al. 1996 J. Therm. Biol. 21(2):101-108). This can have a significant effect on populations of animals such as commercial livestock including cattle, pigs, poultry, horses, and fish, wherein stress can be related to growth inhibition, infertility, and decreased milk or egg production (where applicable) (L. G. Johnson, “Temperature Tolerance, Temperature Stress, and Animal Development”, Proc. Stress Symposia: Mechanisms, Responses, Management. Ed., N. H. Granholm, South Dakaota State University Press, Apr. 6-7, 1993; J. J. McGloner, “Indicators Of Stress In Livestock And Implications For Advancements In Livestock Housing”, Proc. Stress Symposia,: Mechanisms, Responses, Management. Ed., N. H. Granholm, South Dakaota State University Press, Apr. 6-7, 1993; T. Molitor and L. Schwandtdt, “Role Of Stress On Mediating Disease In Animals”, Proc. Stress Symposia: Mechanisms, Responses, Management. Ed., N. H. Granholm, South Dakaota State University Press, Apr. 6-7, 1993; M. J. C. Hessing et al, “Social Rank And Disease Susceptibility In Pigs”, Vet Immunol. Immunopath 43:373-387, 1994; F. Blecha, “Immunoligcal Reactions Of Pigs Regrouped At Or Near Weaning”, Am. J. Vet. Res. 46(9): 1934-1937, 1985; D. L. Thompson et al., “Cell Mediated Immunity In Marek's Disease Virus-Infected Chickens Genetically Selected For High and Low Concentrations Of Plasma Corticosterone”, Am. J. Vet. Res. 41(1):91-96, 1980; Kehrli, H. E. et al., 1989a & b, Am. J. Vet. Res. 50(2):207 and 215).

Thus an animal with a predicted EBV, and thus with a predicted level of immune response, disease resistance or susceptibility, and/or productivity may also have predicted stress coping abilities. For this application, it is preferred that in the methods of the invention the antibody and CMIR traits are determined when the animal is under stress.

As stated above “stress” is any acute or chronic increase in physical, metabolic, or production-related pressure to the animal. It is the sum of the biological reactions to any adverse stimulus, physical, metabolic, mental or emotional, internal or external, that tends to disturb an organisms homeostasis. Should an animal's compensating reactions be inadequate or inappropriate, stress may lead to various disorders. Many events can place an animal under stress. These include, but are not limited to: parturition, weaning, castration, dehorning, branding, social disruption, change in ration, temperature and exercise. Examples of social disruption include, but are not limited to: change of location, shipping, co-mingling and addition or removal of animals from immediate environment.

Growth Hormones

In another embodiment of the present invention, animals with high immune response have increased levels of plasma growth hormone. Thus, these animals may have increased growth and longevity attributes and all other benefits correlated with high levels of growth hormone.

Other Applications

A person skilled in the art can appreciate upon reading the present disclosure, that the methods of the present invention can be used for a number of purposes. The methods can be beneficial in husbandry, in so far as they can be used to influence farming practices and the management of resources. Selecting animals with predicted EBVs can enhance productivity, for instance animals with high EBVs have been found to grow faster and thus reduce the days to market. The growth of the high EBV animals was not due to an increase in the amount of backfat (animals with high EBVs showed no difference in backfat thickness compared to other animals) therefore tissues other than fat must have been growing faster to allow these animals to reach market weight in a shorter amount of time. This suggests that selection for high EBV animals may also provide animals with more lean meat. Further one can select for animals with high EBVs to reduce the requirement of prophylactic and therapeutic treatments thereby reducing the risk of residual prophylactic and therapeutic materials in animal products. Selected animals with high EBVs may also have reduced susceptibility to those infections such as salmonella, camphylobacter, listeria and others which are zoonotic, or transmissible to man. In this way, the selected animals provide products for human consumption with reduced risk of compromising human health due to zoonotic infection The following non-limiting examples are illustrative of the present invention:

### EXAMPLES

The details of the Examples may be modified to accommodate various species but the underlying principles would remain unaltered. The “system”, as used herein unless otherwise indicated, refers to the computer program used in the method of the invention. The specific program used here was an enhanced “Swine Tyme”® program. Any other suitable program could be employed.

### Example 1

### Selection program for selecting and producing animals (eg pigs) having a predicted level of immune response, disease resistance or susceptibility, and/or productivity.

A. Objectives

The objective in this example was to select 3 breeding lines of pigs (eg. Yorkshire, Landrace and Duroc) for High Immune Response (HIR) and other economically important traits (eg. backfat, days to 100 kg, litter size). Thames Bend Farms Ltd (TBF) was the commercial pig breeding company in which this method was utilized. A person skilled in the art would understand that any commercial breeder could be substituted for TBF.

B. Selection Methods

A method for selecting HIR animals, in this case pigs, is described. Immune response (IR) testing began when piglets were approximately 5 weeks of age and required 21 days to complete. Two separate tests were performed, one to evaluate antibody (Ab) and the other to assess cell-mediated immunity (CoM).

An example of the number of pigs required in the nucleus herd and the number of piglets tested and selected during the selection program (eg at TBFL) are outlined in Table 1. Nucleus sows are defined as sows producing purebred litters with tested progeny.

Testing for Immune Response (IR), in this case, was based on performing 60 tests per week. This number may vary depending on the available testing resources.

The number of pigs chosen for IR and performance testing per week and the culling of animals during the selection process are described in Table 2. For example, 39 litters were produced on average per week from the TBF nucleus herd (17 Yorkshire, 9 Landrace, 13 Duroc). The parents of the initial test litters were selected using conventional breeding methods which are based on production traits. After the initial screening for HIR parents (denoted generation 0), parents of tested litters were selected based on the selection index (SI) which is described in general below and in detail in Example 2.

From the 39 litters produced each week, Thames Bend Farms (TBF) selected 12 Yorkshire, 7 Landrace and 9 Duroc litters in which they found at least one male and one female acceptable for HIR testing, inclusion in the nucleus herd, and which represented the better litters from which to select (on the basis of SI, physical soundness, parentage, etc . . . ).

One male and at least three females were kept from each litter for Record of Performance (ROP) testing. The selected male from each litter and one of the three females were IR tested. Other piglets in the litter were not considered further.

After IR testing, the lower 40% of males ranked in descending numerical order according to their preliminary SI (based on performance testing of their relatives) and their estimated breeding values (EBVs) of IR, known as their immune response index (IRI), were removed as HIR candidates. All remaining gilts and boars were ROP tested and a final production index (PI) calculated. The SI was then calculated using the IRI and PI. Males and females were then selected as nucleus replacement stock based on their final SI. For example, following ROP testing, all HIR candidate males (top 60%) not culled had been IR tested. Two thirds of the HIR candidate females had not been IR tested, but have IR EBVs based on information from relatives. The number of animals selected as nucleus replacements are shown in the last row of Table 2. Females were selected every week from a 3 week pool, and males every 4 weeks from a 4 week pool of HIR candidate pigs.

A computer system designed to identify and track all pigs selected for ROP and IR testing automatically indicated the rank of pigs based on IR, PI and SI. This system indicates candidate HER pigs for breeding based on the final SI ranking. The commercial breeding facility selects pigs with the highest SI for breeding. The selection pressure determines the percentage of male and female pigs with the highest SI ranking to be selected for breeding and from this group the final selection decision takes into account (in addition to SI):

C. Monitoring the Relationship Between Immune Response Traits and Other Parmeters

The relationship between IR traits, the IRI and performance traits (age at IR testing, ROP age, backfat and lean depth measurements) was monitored periodically as data were accumulated.

The relationship between IR traits and response to vaccines was also evaluated based on periodical trials.

Data on health and sow productivity were collected routinely in the nucleus herd and, from samples of Fl sows and market hogs, to permit further investigation of the relationships of these traits with IR.

A control line of, for example, 200 Yorkshire sows was also maintained. The control line was not selected for HIR but was selected for performance traits with the same intensity as the HIR Yorkshire line.

TABLE 1
Assumptions for the selection phase

| Assumptions | Yorkshire | Landrace | Duroc | Yorkshire |
|---|---|---|---|---|
| Number of | 400 | 200 | 300 | 200 |
| nucleus sows | | | | |
| Approximate | 17 | 9 | 13 | 9 |
| number of litters | | | | |
| per week | | | | |
| Number of | 39 | 26 | 39 | 26 |
| replacement boars | (23 | (17 | (17 male/ | (17 |
| required per year | male/female) | male/female) | female) | male/female) |
| (if boars are | | | | |
| replaced every | | | | |
| farrowing period, | | | | |
| or whenever | | | | |
| matings for 15-18 | | | | |
| litters are | | | | |
| completed) | | | | |
| Number of | 293 | 147 | 220 | 147 |
| replacement gilts | | | | |
| required per year | | | | |
| (if sows are | | | | |
| replaced after 3 | | | | |
| litters) | | | | |
| Number of gilts | 352 | 176 | 264 | 176 |
| to select per year | | | | |
| (if 20% of | | | | |
| selected | | | | |
| gilts are culled for | | | | |
| breeding reasons) | | | | |

TABLE 2
Actions for the selection phase

| | | | | Control |
|---|---|---|---|---|
| | Yorkshire | Landrace | Duroc | (Yorkshire) |
| Approximate number of litters | 17 | 9 | 13 | 9 |
| per week | | | | |
| Litters selected for HIR and/or | 12 | 7 | 9 | 7 |
| performance testing | | | | |
| Selection for IR testing | | | | |
| 1 male from each litter | 12 male | 7 male | 9 male | 2 male^{d} |
| (cull all other males) | | | | |
| 1 female from each litter | 12 female | 7 female | 9 female | 2 female^{d} |
| (keep at least 2 other females for | | | | |
| ROP testing) | | | | |
| Cull half the IR tested boars on SI | 5 male | 3 male | 4 male | 4.5 male |
| plus thresholds after IR testing | culled | culled | culled | culled^{a} |
| ROP all remaining animals | 7 male | 4 male | 5 male | 2.5 male |
| (including females not tested for | 39 female | 21 female | 30 female | 21 female^{b} |
| IR) | | | | |
| Select male and female with top | 3 male/4 wks | 2 male/4 wks | 3 | 2 male/4 wks^{c} |
| SI for nucleus breeding | | | male/4 wks | |
| | 7 | 3-4 | 5 | 3-4 |
| | female/wk | female/wk | female/wk | female/wk^{c} |

^{a}culled at random by the system among the 7 designated males (1 male per litter)
^{b}8.5 of these females on average were eliminated at random from the selection pool
^{c}selection on DLI only
^{d}from 2 litters picked at random by the system among the 7 designated litters.

### Example 2

### Estimation of breeding values for immune response indicator traits and how that can be combined with estimated breeding values for production traits (eg using pigs).

A. Introduction

Estimated Breeding Values were determined from phenotypic records on individuals and their relatives. The extent to which individuals have genes in common is the same as having breeding values in common. The degree to which genes in common dictate the phenotype is termed heritability (h2). The relative importance of having genes in common is determined from the ratio between the total phenotypic variation (Vp) of a trait to the additive genetic variation (Va). In simple terms, the ratio Va/Vp is heritability. It is essential to know h2 of a trait in order to predict how it will respond to selection. The details of how breeding values were determined for IR traits were described below.

For the case in which pigs were IR tested, EBVs for IR took into account the effect of sex of the animal, the contemporary group in which the individual was tested, and the litter in which it was born. EBVs for IR were based on 2 traits, one which was an indicator of antibody (eg. antibody response following the specified immunization with HEWL) and the other which was an indicator of cell-mediated immune response (eg. DTH response following the specified immunization and subsequent interdermal injection of PPD). Both IR traits, their heritabilities, the genetic variances and phenotypic standard deviations form the bases of the IRI described herein. The IRI was designed to give equal weight to the 2 IR traits, but this can be modified to emphasize one trait above the other if desired in future generations of selection. Currently, in order to ensure that only animals that are superior for both IR traits were selected for breeding, the IRI restricts the selection of animals which were only favourable for one of the traits (antibody or CMI) by imposing thresholds for each IR trait. For example, if an animal ranked at the top of the IRI, but was in the bottom 25% for one of the traits the animal was removed from the selection. This procedure is similar to using independent culling levels to identify individuals with superiority in more than 1 trait.

It was possible to include the IRI with production information by combining the IRI with PI. For pigs, the PI may include EBVs for growth, backfat, litter size, and carcass assessment. The final selection was based on IRI and PI. It was possible to place varied emphasis on immune response traits or production traits by providing different weights to each trait in the index. These weights were generally expressed in terms of estimated dollar values for each trait in the index, and may be altered to suit the value to be placed on immune response or production during the selection. In the example described herein, the economic values were selected to give equal emphasis to immune response and production. Adding information on IR to production indices already in commercial use is expected to further enhance production gains through improvements in health and physiological parameters.

The two IR traits were denoted as PPD and HEWL. The calculation of a selection index from the raw data involved the following steps:

1) scale transformation of the IR data

2) calculation of EBV for IR traits

3) calculation of the IR index (i.e. an index of the EBV for each of the two IR traits)

4) calculation of the overall selection index (which combines the IR index with the conventional production index).

The steps are detailed below, followed by a section on independent culling levels.

B. Scale Transformation of the IR Data

In a previous selection experiment, no scale transformations were carried out. The analysis of data from that experiment resulted in the recommendation that PPD should be log-transformed and HEWL should be analyzed without transformation. This was the approach taken herein. The approach may be altered if analysis of data in the future suggests any other more suitable transformation.

A small study (using the first 100 records in each breed) was done to determine the transformation most effective in removing any relationship between the variance and the mean and normalizing the distribution. The data was divided up into classes at random, and the class standard deviations was regressed on the class means. The log transformation took the general form Y=ln(X+a/b) where a and b are the intercept and slope respectively, of the regression. Details are given in Appendix 1.

Tests of normality and relationship between variance and mean were carried out on the transformed data, as well as a study of the residual relationship between the resulting standard deviation and the mean.

C. Calculation of EBV For IR Traits

All data received from the University of Guelph was used for genetic evaluations.

EBV was calculated based on the following univariate animal model for each trait:

where

yᵢⱼₖₗ=μ+sᵢ+mⱼ+cₖ+aᵢⱼₖₗ+eᵢⱼₖₗ

yᵢⱼₖₗ is the record on pig 1 of sex i and within litter k and contemporary group j

μ is the mean

sᵢ is the fixed effect of sex i

mⱼ is the fixed effect of contemporary group j

cₖ is the random effect of litter k, distributed (0,Iσ²_{c})

aᵢⱼₖₗ is the random effect of the breeding value of animal 1 within sᵢ, mⱼ and cₖ, distributed (0, Aσ²ₐ) where A is the full relationship matrix

eᵢⱼₖₗ is the random residual distributed (0,Iσ²ₑ)

The EBV is the estimated value of aᵢⱼₖₗ. Management groups were groups of pigs tested in the same room and building in the same week. Litters can be cross-classified with management groups. The variance components assumed for the two traits, as proportions of the total phenotypic variance, was as follows:

| PPD | HEWL |
|---|---|
| σ²ₐ = 0.16 | σ²ₐ = 0.27 |
| σ²_{c} = 0.29 | σ²_{c} = 0.27 |
| σ²ₑ = 0.55 | σ²ₑ = 0.46 |

The variance components as proportions of phenotypic variance, were unchanged by any data transformation. Only the phenotypic variance was changed.

The univariate model assumes that the two traits are uncorrelated to each other. As more data accumulates, more accurate estimates of covariance components may be obtained and a two-trait model used instead.

D. Calculation of the IR Index

The index was designed such that when the top animals are selected on index value, their average superiority for HEWL EBV is the same as it is for PPD EBV, when both traits are expressed in terms of phenotypic standard deviation units.

To satisfy this requirement, the index is:

$${I}_{IR} = \frac{{σ}_{P , PPD}}{{h}_{PPD}^{2} {σ}_{A , PPD}^{2}} \cdot {EBV}_{PPD} + \frac{{σ}_{P , HEWL}}{{h}_{HEWL}^{2} {σ}_{A . HEWL}^{2}} \cdot {EBV}_{HEWL}$$

where h², σ²_{A} and σ_{P} denote heritability, genetic variance and phenotypic standard deviation respectively, and the subscripts PPD and HEWL indicate the trait to which the parameter applies.

Heritabilities of 0.27 for HEWL and 0.19 for log(PPD) were previously determined. In data received by The Canadian Centre for Swine Improvement (CCSI), phenotypic standard deviations were 0.45 for HEWL and 0.19 for log(PPD). The index weights were calculated using these values. This provided an index of:

I_{IR}=141EBV_{logPPD}+30.2EBV_{HEWL}

Given the assumptions of the genetic evaluation model in section C, with selection on this index, the average outcome was equal response in phenotypic standard deviations of the two traits.

E. Calculation of the Selection Index

For the two dam lines (ie the Yorkshire and Landrace breeds), the production index was the dam line index, which combines EBV for backfat (EBV_{FAT}), age at 100 kg weight (EBV_{AGE}) and litter size (EBV_{NB}):

DLI=$1.54EBV_{NB}−$0.46EBV_{FAT}−$0.11EBV_{AGE}

with a phenotypic standard deviation of $5.02.

For the Duroc breed the production index was the sire line index which combines only backfat and age at 100 kg weight:

SLI=−$0.92EBV_{FAT}−$0.22EBV_{AGE}

with a phenotypic standard deviation of $3.40.

The sire and dam line indices were expressed in terms of profit per market pig, in a production system using Fl dams (from the two dam lines) and terminal sires (from the sire line).

The selection index assumes that an increase of one phenotypic standard deviation in the IR index produces the same increase in profit per hog as an increase of one phenotypic standard deviation in the production index.

If the IR index is I_{IR}=141EBV_{logPPD}+30.2EBV_{HEWL}, then its phenotypic variance is:

σ_{p,IR}=_(141²(0.19²)+30.2²(0.45²)) =30.4

For Yorkshire and Landrace, the selection index is:

SI=(DLI/5.02)+(I_{IR}/σ_{p,IR}),

and for Duroc it is

SI=(SLI/3.40)+(I_{IR}/σ_{p,IR}).

Thus it was assumed that a phenotypic standard deviation of the IR index in Yorkshire and Landrace, is worth $5.02 increased profit per market hog. In Duroc, the sire line, it was assumed that a phenotypic standard deviation of the IR index is worth $3.40 increased profit per market hog.

The indexes can be expressed in dollar values by multiplying by 5.02 for Yorkshire and Landrace:

SI=$1.54EBV_{NB}−$0.46EBV_{AGE}+$0.11EBV_{AGE}+$23.28EBV_{log(PPD)}+$4.99EBV_{HEWL}

and by 3.40 for Duroc:

SI=−$0.92EBV_{FAT}−$0.22EBV_{AGE}+$15.77EBV_{log(PPD)}+$3.38EBV_{HEWL}

The improvements across lines were assumed to be additive (ie the overall effect of IR selection on profit per market hog is the sum of the contributions from Yorkshire, Landrace and Duroc).

The economic values (i.e. the estimated effects on profit) for IR are arbitrary in the absence of any data on the profitability of market hog production from lines with different IR status.

The values may be estimated based on data collected from on-going experiments.

F. Independent Culling Levels

It has been suggested that animals should be selected only if they meet a minimum threshold for each IR trait. This corresponds to the use of independent culling levels.

For the time being, selection was based on culling of pigs in the bottom 25% of the population on EBV for either IR trait, followed by index selection among the remaining animals. Reports were produced however, to show all animals ranked by selection index, in order to observe the effect of independent culling levels on selection.

As more data becomes available from the testing phase, a more efficient procedure may be developed (Appendix 2).

In practice, with selection among small numbers of animals on-farm, the threshold EBV above and below which the top and bottom 25% lie must be defined. For now it was assumed that the standard deviation of EBV is 0.122 for HEWL and 0.0367 for log(PPD). This means that the thresholds, based on the normal distribution and a mean EBV of zero, are +/−0.082 for HEWL and +/−0.025 for log(PPD). Thus pigs which have EBV outside this range were predicted to be in the top or bottom 25%.

Because not all pigs were HIR tested, the standard deviation of EBV may be lower than assumed here, but the thresholds being assumed were checked as data accumulated.

G. Summary of Selection Indexes and Repeatabilities

Index for Yorkshire and Landrace:

SI=$1.54EBV_{NB}−$0.46EBV_{FAT}−$0.11EBV_{AGE}+$23.28EBV_{log(PPD)}+$4.99EBV_{HEWL}

Repeatability

=0.261REP_{NB}+0.059REP_{FAT}+0.066REP_{AGE}+0.452REP_{log(PPD)}+0.162REP_{HEWL}

Index for Duroc:

SI=−$0.92EBV_{FAT}−$0.22EBV_{AGE}+$15.77EBV_{log(PPD)}+$3.38EBV_{HEWL}

Repeatability

=0.302REP_{FAT}+0.337REP_{AGE}+0.266REP_{log(PPD)}+0.095REP_{HEWL}

where REP_{NB}, REP_{FAT}, REP_{AGE}, REP_{log(PPD)}, and REP_{HEWL} are the repeatabilities of the EBV.

H. References

Kleczkowski, A (1949) The transformation of local lesion counts for statistical analysis. Ann. Appl. Biol. 36:139-152

Pirchner, F (1983) Population Genetics in Animal Breeding. 2nd Ed., Plenum Press, NY.

### Example 2

### Appendix 1 SCALE TRANSFORMATION

The data can be divided up into classes at random, and the class standard deviations regressed on the class means. If the regression is roughly linear, the log transformation Y=log (X+a/b) will render the standard deviation on the transformed scale independent of the mean (Kleczkowski, (1949), where a is the intercept and b is the regression slope. (The simple transformation Y=log(X) does this only if the intercept of the regression is zero).

Scale transformations improve the accuracy of the EBV where the variance depends on the mean, where the data has a skewed distribution, or where there are nonadditive interactions. The second two problems are often related to the first. For example, when the data is divided into groups, and groups with higher means have higher variances, this automatically produces positive skewness in the overall data when the groups are combined. Hence a transformation derived with the objective of removing relationships between mean and variance, can also reduce the other problems.

If the regression of the standard deviation on the mean is significantly non-linear, a polynomial regression can be used, and the appropriate transformation is a function of the regression equation as shown below:

If the transformation is Y=f(X), where X is the raw data, then by a single term Taylor expansion:

Y=f(μ)+f′(μ)(X−μ),

where μ is the mean on the raw scale. If E[ ] denotes expectation, then E[X−μ]=0, and so

E[Y]=E[f(μ)]=f(μ).

and the expectation of the variance on the transformed scale is:

$$\begin{matrix}E [ Var ( Y ) ] = E [ {( Y - f ( μ ) )}^{2} ] \\ = E [ {( {f}^{′} ( μ ) ( X - μ ) )}^{2} ] \\ = {( {f}^{′} ( μ ) )}^{2} \cdot E [ {( X - μ )}^{2} ] \\ = {( {f}^{′} ( μ ) )}^{2} \cdot Var ( X ) \\ = {( {f}^{′} ( μ ) )}^{2} \cdot g ( μ )\end{matrix}$$

where g(μ) is the variance of the raw data (Var(X)) as a function of the mean (μ). We want E[Var(Y)] to be independent of μ, and so we require that:

f′(μ)=1/√(g(μ))

=>f(μ)=f(g(μ))^{-0.5} dμ

The best-fit regression of class variances on class means is used as g(μ), and the variance stabilizing transformation f(μ) is determined by integrating with respect to μ as shown in the equation directly above.

Generally if g(μ)=μⁿ, (n any number except 2) then the transformation is:

f(X)=X^{-0.5n+1}.

Examples are:

| | | |
|---|---|---|
| g(μ) = μ² | =>log transformation, | f(X) = ln(X) |
| g(μ) = constant | =>no transformation, | f(X) = X |
| g(μ) = μ (linear regression) | =>square root transformation, | f(X) = √X |

### Example 2

### Appendix 2 ECONOMIC SELECTION INDEXES VERSUS INDEPENDENT CULLING LEVELS

If the traits being selected are controlled by effects at many genetic loci, each of small effect, then selection on the index gives rather more genetic improvement than the use of independent culling levels. For example, with 2 uncorrelated traits with the same heritability and economic value, and 10% of the animals selected, index selection gives 10% more genetic response than independent culling levels (eg Pirchner, 1983, pl96).

If the traits are negatively correlated, the advantage of index selection increases. Independent culling levels have an advantage only where the traits are influenced by major genes or where there is a limitation in the selection index, such as economically important traits being missing (eg conformation or physical soundness), or economic values of traits being incorrect.

Although the IR index used was a linear index, there is some expectation that the two IR traits have a synergistic action such that their effect on disease incidence is nonadditive.

In this case the theoretically best procedure is to estimate the non-linear profit function where the IR index must include a positive interaction term:

I_{IR}=k₁EBV_{PPD}+k₂EBV_{HEWL}+k₃EBV_{PPD}*EBV_{HEWL}

With a linear index, a pig which is +3 for one trait and +1 for the other might be equal to a pig which is +2 for both traits. With a non-linear index, such as that shown above where k₃ is a positive weight, the pig which is +2 for both traits has a higher index and is preferentially selected. Use of the non-linear index has some apparent similarity to independent culling levels, but gives better genetic response in disease resistance if the profit function is estimated correctly.

The profit function could be estimated from the relationship between IR and economic traits in the testing phase, and then used to derive a more accurate IR index.

### Example 3

### Selection and culling procedures of animals in selected lines

This example describes the procedures for the selection and culling of animals (eg pigs) in HIR selected lines.

A. Selection of Piglets Within Litters

The system produced for each breed a weekly list of litters from which to select piglets for testing.

On average, based on the size of the nucleus for each breed, there was 17 Y, 9 L and 13 D litters per week in the nucleus. TBF designated among these 12 Y, 7 L and 9 D litters that have at least one pig of each sex acceptable for IR testing and selection.

The extra litters in each breed were the “reserve” litters.

At 3-5 days of age, one male was kept from each of the designated litters. Other males were castrated.

At weaning, TBF designated which pigs to IR test and performance test. As a rule, the selected male in each litter was IR and performance tested. Also, 3 females from each litter were chosen for performance testing, and one of those was chosen for IR testing.

TBF decided which piglets to keep and which piglets to IR test in each litter. The choice was based on physical soundness, size and conformation (legs, underline), for example.

If there were not enough piglets of one sex to select in a litter, TBF indicated the reasons on the list and selected more from other litter(s). However, as a rule, the number of piglets selected from one litter did not exceed 2 males or 4 females, and those IR tested in one litter did not exceed 2 males or 2 females.

If not enough pigs of one sex were available to IR or performance test from all the designated litters in a breed, TBF was able to use pigs from “reserve” litters. As much as possible, the use of reserve litters was kept to a minimum.

If in a given week the use of reserve litters was not enough to achieve the projected number of IR tests in one breed and sex, more IR tests from the other sex or from another breed were made in that specific week, and the reverse carried out the following week so that the weekly target was reached over an average of two weeks.

B. Producing Weekly Reports After HIR Testing

EBVs for MIR were computed by CCSI each week for all remaining males and females in each litter following IR testing. The system used these EBVs along with pedigree EBVs for production traits to compute selection indices for these animals.

Each week, after the selection indices were computed, the HIR inventory report for all animals IR tested during the last week was generated (report # 1). The report was sorted by breed, sex and SI, regardless of selection status code.

As shown in Table 2 of Example 1, there should be 56 pigs IR tested per week (24 Y, 14 L and 18 D), with the same number of each sex.

The report was checked to ensure that all animals tested were present, that they had reasonable EBVs and S's, that contemporary groups were as expected, etc . . .

C. Weekly Culling of IR Tested Males

After report # 1 was generated, the weekly selection and culling report for males was generated (report # 2).

When report # 2 was generated, the system identified males to cull (castrate) at 9-10 weeks of age. This included males in the bottom 25% of the population for either IR trait plus any remaining males with low SI (selection indices). Approximately half of the males were culled (5 out of 12 per week for Y, 3 out of 7 for L, 4 out of 9 for D).

Report # 2 listed all males (kept and culled) IR tested this week, by breed and SI.

TBF was provided with a list of males to cull this week (those with a status code of “C” in the above report).

D. Producing Weekly Reports for Performance Tested Pigs

Each week, for the 3 breeds selected, there were about 16 males performance tested (all IR tested).

Each week, for the 3 breeds selected, there were about 90 females performance tested (⅓ of which were IR tested).

Each week, CCSI computed EBVs for IR traits for these animals. The system used these, along with on-farm EBVs, to compute selection indices.

Each week, the HIR inventory report was generated for all animals probed during the last week (report # 3). The report was sorted by breed, sex and SI.

The report was checked to ensure that numbers of animals, contemporary groups, EBVs and SI's were as expected.

E. Weekly Culling of Performance Tested Males

After report # 3 was checked, report #4 was generated. Report #4 was a special version of the Selection and Culling report which assigned cull codes to performance tested males based on SI and IR thresholds. Since a new male selection pool was formed every 4 weeks, and its maximum size was about twice the number of new boars per week, most of the culling occurred in the 3^{rd} and 4^{th} week. Report # 4 showed all males kept in the pool and those to cull this week.

TBF was provided with the list of males to cull (those with a status code of “C” in report # 4).

F. Weekly Selection and Culling of Performance Tested Females

The selection of females was carried out every week from a 3 week-selection pool.

To do the selection, report # 5 was generated (selection and culling report for females).

As report # 5 was generated, the system culled the bottom end of the 3-week female selection pool, and preselected 10 Y, 6 L and 8 D females from this pool, based on SI and HIR thresholds. The report showed preselected females by breed and SI. On average, the proportion of preselected females over selection candidates was 25.6% for Y, 28.6% for L, and 26.6% for D, assuming very few top SI females were culled because of IR thresholds.

TBF used this report to select an average of 6.8 Y, 3.4 L and 5.1 D each week. TBF may also have culled some preselected females if they were found unacceptable (then they did not appear again in the selection pool). The “select animal entry” input window was used to enter these selections into the system. Once all females were selected, as the window is closed, the system culled any females in the pool that had not yet been selected or culled and had been probed more than 3 weeks ago.

TBF was provided with a report of all females culled this week from the project, so they could be bred for purposes other than HIR.

Some females were neither selected nor culled for a period of up to three weeks, but they were not normally ready to breed before then.

Selected females were included in the weekly list of selected HIR nucleus females to breed. The list included selected sows and gilts that were ready for breeding that week.

Selected sows were taken off the list after their third litter (or sooner if a decision was made to increase the replacement rate).

G. Monthly Selection and Culling of Performance Tested Males

The selection of males was carried out every four weeks, after all weekly reports were generated (especially report # 4).

To do the selection, report # 6 was generated. When report # 6 was generated the system preselected 5 Y,3 L and 4 D males from the pool of boars accumulated over the previous 4 week period, based on SI and IR thresholds. The report showed preselected males by breed and SI. On average, the proportion of males selected over selection candidates was 10.4% for Y, 10.7% for L and 11.1% for D, assuming very few top SI males were culled because of IR thresholds or because of pre-selection.

TBF then used this report to select 3 Y, 2 L and 3 D on average each month. The “select animal entry” input window was used to enter these selections into the system. As the window was closed, indicating the end of selections for this month, the system assigned cull codes to all unselected males except for 1 reserve boar per breed (the unselected boar at the top of the breed), and produced a report of males to cull.

The selected males were included in the list of “HIR nucleus males available for breeding”.

There was a small cost to keeping intact some preselected males for up to 4 weeks. However, the number of boars involved was small and preselected boars could be used for multiplication and other purposes while awaiting selection.

H. Use of Selected Males

TBF endeavoured to use boars to produce no more than 17-23 litters per boar, so as to equalize the use of boars across females.

Selected boars that had been used for more than 2 months or had produced more than 23 litters were flagged by the system for culling.

Only males and females selected through the above procedures were used to produce the next generation of nucleus males and females.

The females in the list of “HIR nucleus females to breed” were bred to males in the list of “HIR nucleus males available for breeding”.

TBF decided which available males to mate with which available females, taking into account trait complementarity (e.g. correction of physical defects), the need to maintain inbreeding at a reasonable level, and the need to use boars in a roughly equal way across available females (target of 17-23 breedings per sire).

I. Monthly Monitoring Report

Every 4 weeks, the HIR inventory report was used to list all animals HIR and/or performance tested during the last 4 week period, sorted by breed, sex and SI, along with their appropriate testing and status codes. This included animals with blank, preselected, selected, reserve or override selection status codes.

### Example 3

### Appendix 1

### Reports for selection and culling—Summary

A. Format

The “HIR Inventory list” and “Animal Selection and Culling” report have the same format. However, they are functionally different, since the latter is used as a way to make the system carry out various tasks (assign preselected codes and cull codes, for example).

The common report format contains most of the performance and HIR information that can influence selection decisions. A description of the fields included in these reports and their order of appearance is given in Appendix 2.

B. Production of Routine Reports

All reports below were generated routinely. Reporting options are pre-set so they remain the same over time.

1. Weekly Reports

Last Week's HIR Results (Report # 1)

HIR inventory report for all animals HIR tested during the last week, by breed, sex and SI (regardless of selection status code).

Culling of Last Week's HIR Tested Males (Report # 2)

When the selection and culling report is run for these males, the system will assign a selection status code of “culled” to the lower half of the animals based on SI and IR thresholds. The selection and culling report is then produced, listing all males (kept and culled) this week, by breed and SI.

Last Week's Performance Results (Report # 3)

Each week, after performance testing and EBV computation, produces a list of all animals performance tested in the last week, sorted by breed, sex and SI, using the HIR inventory report.

Weekly Culling of Performance Tested Males (Report # 4)

As the Selection and Culling report is generated, the system assigns cull codes to males based on SI and IR thresholds. The Selection and Culling report shows all males kept in the pool and those culled this week.

Weekly Selection and Culling of Females (Report # 5)

As the Selection and Culling report is generated, the system will cull the bottom end of the female selection pool and assign “preselected” codes to the top 25% of remaining females. The Selection and Culling report shows only preselected females, sorted by breed and SI.

TBF then uses the report to select females each week. Codes for selected females (and any additional comments) can be entered into the system using the “select animal entry” input window. Once all females for the week have been selected, as the window is dosed, the system will cull any females in the pool that have not yet been selected or culled and have been probed more than 3 weeks ago. A report of all females culled this week from the project is produced, so they can be bred for purposes other than HIR.

2. Four Week Reports

These reports are run every 4 weeks after the reports for the current week have been produced.

Selection and Culling of Performance Tested Males (Report # 6)

As a special version of the Selection and Culling report is generated every 4 weeks, the system preselects the top males in the 4 weeks boar pool, keeping the numbers shown in Table 1 (of Example 1) plus one reserve boar per breed, based on SI and thresholds. The Selection and Culling report shows only preselected and reserve males, sorted by breed and SI.

TBF then uses the report to select boars for this month, and uses the “select animal entry” input window to enter these selections into the system. As the window is closed, indicating the end of selections for this month, the system assigns cull codes to all remaining males and produce a report for additional males to cull.

Monitoring Report (Report #7)

Every 4 weeks, the HIR inventory report is used to list all animals HIR and/or performance tested during the last 4 week period, sorted by breed, sex and SI, along with their appropriate testing and status codes. This includes animals with blank, preselected, selected, reserve or override selection status codes.

### Example 3

### Appendix 2

### Suggested format for the HIR inventory and Selection and culling reports

Breed (4 ch. Max)

Tag

Tattoo

Sex

Birth date

Sire tattoo

Dam tattoo

Genetic line (4 ch.)

Testing code (T, or blank if untested)

Barn number (HIR testing)

Contemporary group number (HER testing)

Probe date

Selection status code (blank, P, C, S, R or O)

Adj age

Adj fat

EBV age

EBV fat

EBV # born (leave blank for Duroc)

REP of above

DLI (for Y, L) or SLI (for D)

REP of above

EBV PPD

Threshold indicator (*)

REP of above

EBV HEWL

Threshold indicator (*)

REP of above

SI

REP of above

### Example 3

### Appendix 3

### Instructions for programming selection and culling procedures and reports

1. Male Selection

First Stage (After IR Testing)

CCSI computes SI of animals as soon as IR testing is done and contemporary group is complete (lower minimum contemporary group size to 14 to allow Landrace groups to fill up in 1 week).

Each week, system culls bottom 40% of all males tested in each breed, based on SI and IR thresholds (see table 2 for exact numbers). This is done by generating report # 2. If the contemporary group is not complete, there may be no animals to cull for that breed.

Second Stage (After Performance Testing)

CCSI computes SI for all animals each week.

In each breed, system sorts by SI all males which have not been selected or culled.

Each week, system culls lower end of these by SI and IR thresholds. The following numbers to be left per breed in the “selection pool”:

Y=12 L=8 D=10

This is done by generating report # 4. In the first week of IR testing, no animals should be selected or culled.

Every four weeks, system preselect and list the following numbers of top animals in the pool by SI i.e.:

Y=5 L=3 D=4

This is done by generating report # 6.

Every four weeks, TBF selects the following average number of males among those listed:

Y=3 L=2 D=3

Every four weeks, system culls every male which was not selected in 5) above except for 1 reserve boar per breed (unselected boar at the top of the pool).

TBF uses selected males quickly in the HIR nucleus once selected, in order to produce about 23 litters per boar in Y and 17 litters per boar in L and D. Afterwards, the boars may be used for other purposes (other lines, multiplication, commercial use). If a selected boar does not work out, the reserve boar are used instead.

2. Female Selection

After new probe records have arrived, SI is computed for all animals.

Per breed, all females from HIR tested litters, which have not been selected or culled, are sorted by SI in the data base These females may or may not have been IR tested themselves.

System culls the lower end of the above females by SI and IR thresholds so that the following number are left per breed:

Y=40 L=24 D=32.

This is done by generating report # 5.

No female is selected or culled until the second week of the selection phase (this is valid for both the system and TBF).

System preselects and lists the top 25% of remaining females by SI. The following numbers will be listed:

Y=10 L=6 D=8

System assigns a preselection code (P) to these females.

TBF selects the following average number of females among preselected females:

Y=6.8 L=3.4 D=5.1

The decimals imply one can select about 3 females one week and 4 the next in the Landrace breed, for example. TBF can also cull preselected females that are unacceptable for selection.

System culls all females that were not probed in the last 3 weeks of probing, including the current one.

3. Animal Status Code

Each animal is assigned a status code in the system, which for each animal can have one of five values:

blank=animal has not been preselected, selected, or culled.

P=animal has been “preselected” by the system and is listed as a selection candidate; this code is assigned by the system when the animals are “listed” (top 25% of pool for females, top 40% of pool for males). This is done in step 4 for females, B4 for males.

S=animal has been selected by TBF, this code is assigned by TBF, not by the system (step 5 for females, B5 for males).

C=animal has been culled, either by the system (steps 3 or 4 for females, A2, B3 or B6 for males) or by TBF (step 5 for females, B5 for males). As a rule, TBF only needs to cull preselected animals that are unacceptable because of conformation or other defects. All other culling is done by the system based on SI or IR thresholds.

O=this stands for “override”, and will be assigned by the system instead of the code “S” if an animal not preselected by the system (i.e. with a code other than P) has been selected by TBF in step 5 for females, or B5 for males. In reports showing selected animals, the code “S” or “O” should be displayed. The override feature allows TBF, in exceptional circumstances, to select animals that were not preselected by the system, but it makes this apparent on selection reports.

A preselected animal (code P) may later be selected by TBF (in which case his code will change to S), or it may be culled by TBF (if TBF judges this animal has serious defects that should prevent it from ever being selected), or it may be left with a P code so that it remains available for selection later. With the process described above, a female with a blank or P code has 3 chances of being selected (3 consecutive weeks) and a male 1 (but from a 4 week pool). Afterwards, the animal is automatically culled from the project as per steps 6 or B6.

In step 4 for females and B4 for males, the preselection codes are reassigned for all animals in the selection pool, i.e. the top 25% of females or 50% of males are given a P code, while the others are given a “blank” code, even if they had a P before. This reflects the fact that an animal preselected in a given week, but not selected or culled by TBF, will only remain preselected the next week if he is in the top % of the new selection pool (after a new week of animals has been added). Many preselected animals might be culled in steps 3 or B3 because their SI is not high enough to make the new selection pool.

### Example 4

### Selection and culling procedures of animals (eg pigs) in the control

A. Objective

To provide a standard against which to measure the effect of selection for HER on genetic change for economically important traits, separately from other factors.

B. Principles

The control line is not selected for HIR. However, it is selected for production traits with the same intensity as in the selected line.

The control and the selected line are placed in the same management conditions.

As a result, differences between the selected line and the control line for the traits of interest reflect only the effect of selection for HIR over successive generations.

The traits of interest include IR traits, production traits (litter size, age, backfat) and any other traits which can be measured but are not selected (response to vaccines, incidence and cost of health related events, feed efficiency, female productivity traits other than litter size, etc . . . ).

The differences between the selected and control lines in the IR traits over generations provides a means of measuring the genetic change and realized heritabilities for these traits. Estimates of genetic changes for IR in both lines will also be available from BLUP analysis.

There were 3 selected lines, one for each breed. However, to obtain significant results, one control line is better than several control lines of reduced size. Therefore, the control line was composed of Yorkshire animals, and all comparisons will be made to the selected Yorkshire line.

C. Establishment of the Control Line

The control line was established by randomly selecting female full-sibs of the sows that make up the selected Yorkshire line, or if this proved impractical, by taking a random sample of sows from the same population that gave rise to the selected Yorkshire line. For this purpose, the system picked randomly 17 selected and 9 control litters among 26 Yorkshire litters designated by TBF for the project. This process ceased once litters were available from control gilts mated to control boars, and from selected gilts mated to selected boars.

The control litters originated from matings to the same group of boars as those used to produce the first group of IR tested pigs in the selected Yorkshire line.

Later on, control boars and gilts were mated to each other as per the method described below.

Control animals were mixed in with those of the Yorkshire line, i.e. they were in the same barns and pens so they receive the same treatment.

D. Selection Methods

Because the control line had the same size as the Landrace line (200 sows), the number of litters per week and the number replacement boars and gilts required were the same (see Table 1, Example 1).

Approximately 9 litters were produced per week in the control line. From these, TBF selected 7 where they can find at least one male and one female acceptable for selection, and which in their opinions represented the better litters to select from (on the basis of PI, physical soundness, parentage, etc . . . ).

One male and at least three females were selected by TBF in each litter for further testing.

For the purpose of comparing selected and control lines for IR after each generation, the system picked 2 litters at random among the 7 chosen by TBF. One male and one female from each of these 2 litters were IR tested (total of 4 control pigs per week)

When the 7 males (2 of which were IR tested) reached 9-10 weeks (same age as for selected line males), 4.5 of them were culled at random by the system (4 one week and 5 the next). This reduced the proportion of males selected in the control line to about 30% so that the expected genetic change for production traits from male selection was the same in the control and selected lines.

All 3 females per litter were ROP tested. However, of the 21 tested, 8.5 were culled by the system before they were considered for inclusion in the selection pool ( 8 one week and 9 the rest). Therefore the average number of female selection candidates per week was reduced from 21 to 12.5 in the control line. This reduced the proportion of females selected in the control line to about 48% so that the expected genetic change for production traits as a result of female selection was the same in the control and selected lines.

All other selection procedures, i.e. the size of selection pools for males and females, the number of animals listed, the average number that TBF should select, etc . . . remained the same as for the selected Landrace line

The reports generated for the control line were the same as for all selected lines. However, for the control line exclusively, report # 2 randomly culled 9-10 week old males rather than culling them on SI and IR thresholds. Similarly, report # 5 randomly culled some of the females that had just been performance tested before they were included in the pool. All control line selection afterwards was based on the DLI, rather than IR thresholds and SI.

Control line animals were identified as such throughout the system, and therefore carried a separate code. This was done through additional “project” codes, i.e. project animals were either “selection” or “control”. An alternative would be to create a separate breed code for control animals. Since all control animals will be of the Yorkshire breed, this might be relatively easy to do.

The trends in EBV for production traits were monitored routinely in both the selected and the control lines to check that the rate of progress for these traits was similar. Selection procedures in the control line were then adjusted if necessary.

### Example 5

### Predicted response to selection in sire and dam selected lines (eg pigs) under different selection intensities

A. An index giving one phenotypic standard deviation of response in IRI for each one phenotypic standard deviation of response in SLI (or DLI).

i) Index for Sire Lines

Since the SLI is (−0.92FAT−0.22AGE) and the phenotypic variances of backfat and age are 4.5 mm² and 153 days² respectively, the phenotypic standard deviation of the SLI is $3.35. Since the IRI is (141PPD+30.2HEWL) and the phenotypic variances of PPD and HEWL are 0.0361 and 0.2025 respectively, the phenotypic standard deviation of the IRI is $30.04

The variance of the SLI is assumed to be $1.96 and the variance of the IRI is assumed to be $39.4. Thus an index of (SLI/1.96)+(IRI/39.4) would give an equal dollar response in each component. The index giving equal response in terms of phenotypic standard deviations of each component is:

$$\begin{matrix}SI = ( 3.35 / 1.96 ) SLI + ( 30.04 / 39.4 ) IRI \\ = 1.71 SLI + 0.762 IRI\end{matrix}$$

The index weights in the SLI are economic values in dollars. Therefore an SI expressed in dollars is obtained by dividing the above expression by 1.71:

$$\begin{matrix}SI = SLI + 0.446 IRI \\ = - 0.92 FAT - 0.22 AGE + 62.89 PPD + 13.47 HEWL\end{matrix}$$

ii) Index for Dam Lines

Since the DLI is (1.54 LITTER SIZE −0.46FAT−0.11AGE) and the phenotypic variances of litter size, backfat and age are 9 pigs², 4.5mm² and 153 days² respectively, the phenotypic standard deviation of the DLI is $4.91 (litter size contributes 88% of the phenotypic variance). The phenotypic standard deviation of the IRI is $30.04, as in a) above. The variance of the DLI is assumed to be $0.85 and the variance of the IRI is assumed to be $39.4. Thus the index giving equal response in terms of phenotypic standard deviations of each component is:

$$\begin{matrix}SI = ( 4.91 / 0.85 ) DLI + ( 30.04 / 39.4 ) IRI \\ = 5.78 DLI + 0.762 IRI\end{matrix}$$

The index weights in the DLI are economic values in dollars. Therefore an SI expressed in dollars is obtained by dividing the above expression by 5.78:

$$\begin{matrix}SI = DLI + 0.132 IRI \\ = 1.54 LITTER SIZE - 0.46 FAT - 0.11 AGE + 18.59 PPD + 3.99 HEWL\end{matrix}$$

iii) Responses

Table 3 shows the responses in the individual traits to selection of the top 10% of animals on the SI in a dam line. The responses to selection on the DLI are also shown. For the same selection criterion, the ratios of responses between the traits is constant across selection intensities. Table 4 shows the responses in the individual traits to selection of the top 10% of animals on the SI in a sire line. The responses to selection on the SLI are also shown. In sire lines, the SI puts relatively more weight on the IRI, than it does in dam lines.

B. Response to Selection when ⅔ of females are not tested for HIR traits and the index is designed to give one phenotypic standard deviation of response in IRI for each one phenotypic standard deviation of response in SLI (or DLI).

The variances of the EBV used to calculate the responses in Tables 3 and 4 are the variances of the EBV among tested animals in previous genetic evaluations. In future only ⅓ of the selection candidate females will be tested, so the accuracy and variability of the HIR trait EBV will differ between different selection candidates, depending on whether they are tested, and on whether their dams are tested. There are 4 possible situations (individual and dam both tested, only the individual tested, only the dam tested, and the individual and dam both untested). In a previous report (“Predicted Genetic Improvement in HIR with Selection on an Index of HIR, Backfat, and Age at 100 kg”, August, 2000), repeatabilities of the IRI were calculated for each of the 4 situations, under the assumption that the IRI was a single trait with a heritability of 25%. Table 5 shows the results. An approximation that EBV repeatabilities vary directly with heritabilities was used to obtain the repeatabilities of the PPD and HEWL EBV shown in Table 3. The repeatabilities were averaged across the different situations with respect to test data, to obtain the average repeatabilities shown in Table 5.

It is assumed that the variances of the EBV are:

| backfat: | 1.4 mm² |
|---|---|
| age: | 16 days² |
| litter size: | 0.15 |
| PPD (males): | 0.00178 |
| PPD (females): | 0.00144 |
| PPD (average): | 0.00161 |
| HEWL (males): | 0.0240 |
| HEWL (females): | 0.0191 |
| HEWL (average): | 0.0216 |

The variance of the HIR EBV is higher in males than in females. Therefore if the same index is used in both sexes, there ratio of response in IRI to response in SLI is higher in males than in females.

However, in order to obtain an index which gives close to equal long-term response in IRI and SLI, the average variance will be assumed. Then, an IRI of (141PPD+30.2HEWL) has a variance of 51.71.

i) Indexes for Sire and Dam Lines

In sire lines, the index giving equal response in terms of phenotypic standard deviations of each component is:

$$\begin{matrix}SI = ( 3.35 / 1.96 ) SLI + ( 30.04 / 51.71 ) IRI \\ = 1.71 SLI + 0.581 IRI\end{matrix}$$

The index weights in the SLI are economic values in dollars. Therefore an SI expressed in dollars is obtained by dividing the above expression by 1.71:

$$\begin{matrix}SI = SLI + 0.340 IRI \\ = - 0.92 FAT - 0.22 AGE + 47.94 PPD + 10.27 HEWL\end{matrix}$$

In dam lines, the index giving equal response in terms of phenotypic standard deviations of each component is:

$$\begin{matrix}SI = ( 4.91 / 0.85 ) DLI + ( 30.04 / 51.71 ) IRI \\ = 5.78 SLI + 0.581 IRI\end{matrix}$$

The index weights in the DLI are economic values in dollars. Therefore an SI expressed in dollars is obtained by dividing the above expression by 5.78:

$$\begin{matrix}SI = DLI + 0.100 IRI \\ = 1.54 LITTER SIZE - 0.46 FAT - 0.11 AGE + 14.10 PPD + 3.02 HEWL\end{matrix}$$

ii) Responses

In sire lines, the expected responses to selection of the top 10% of males as shown in Table 6a and expected responses to selection of the top 10% of females as shown in Table 6b. Table 7 shows the same results for dam lines. Table 8a shows the expected overall annual responses to selection in a sire line if 11% of males and 26% of females are selected, and generation intervals are 12 and 18 months in males and females respectively. Table 8b shows the same results for a dam line. Because males are evaluated more accurately for HIR than females, there is relatively more expected response in HIR and less expected response in other traits in males, than in females. Across sexes, Tables 8a and 8b shows that the overall expected responses in IRI and SLI (or DLI) in phenotypic standard deviations are roughly the same, as intended by the index formulation.

C. Response to Selection when ⅔ of females are not tested for HIR traits and the index gives equal economic value to one phenotypic standard deviation of IRI and one phenotypic standard deviation of SLI (or DLI).

For sire lines, the index is:

SI=−0.92FAT−0.22AGE+15.77PPD+3.38HEWL

For dam lines, it is:

SI=1.54LITTER SIZE−0.46FAT−0.11AGE+23.28PPD+4.99HEWL

Assuming the EBV have the same variances as in section B above, the expected responses to selection of the top 10% of males and females in sire lines is as shown in Table 9. Table 10 shows the same results for dam lines.

Table 11a shows the expected overall annual responses to selection in a sire line if 11% of males and 26% of females are selected, and generation intervals are 12 and 18 months in males and females respectively. Table lib shows the same results for a dam line. Selection on the overall SI gives less response in IRI than in SLI (or DLI), and this is because in this index IRI has a smaller variance than SLI (or DLI). The SI used here puts less weight on the IRI traits than the SI in section B above, which gave equal response in MRI and SLI (or DLI).

D. Expected proportions of animals with phenotypic IR indexes above the original unselected mean after 1, 2, 3, 4, and 5 years of selection

These proportions are calculated for the equal response SI and for the equal economic value SI in sections B and C above, both for sire and dam lines. The results are shown in Table 12.

TABLE 3
Responses to selection in a dam line, in phenotypic standard
deviations of each trait or index (Yorkshire and Landrace,
top 10%), when the index is designed to provide equal
expected response for IR and production traits.

| | | |
|---|---|---|
| | | Selection |
| s.d. of EBV | phenotypic | criterion |

| | or index | s.d. | SI | DLI |
|---|---|---|---|---|
| backfat (mm) | 1.18 | 2.12 | −0.43 | −0.73 |
| age (d) | 4 | 12.4 | −0.20 | −0.34 |
| litter size | 0.39 | 3 | +0.11 | +0.15 |
| log(PPD) | 0.0361 | 0.19 | +0.18 | 0 |
| HEWL | 0.1215 | 0.45 | +0.18 | 0 |
| IRI | $0.83 | $3.96 | 0.25 | 0 |
| DLI | $0.92 | $5.02 | 0.25 | 0.38 |

TABLE 4
Responses to selection in a sire line, in phenotypic standard
deviations of each trait or index (Duroc, top 10%), when the
index is designed to provide equal expected response
for IR and production traits.

| | s.d. of EBV or | phenotypic | Selection criterion | |
|---|---|---|---|---|
| | index | s.d. | SI | SLI |
| backfat (mm) | 1.18 | 2.12 | −0.34 | −0.76 |
| age (d) | 4 | 12.4 | −0.16 | −0.35 |
| log(PPD) | 0.0361 | 0.19 | +0.25 | 0 |
| HEWL | 0.1215 | 0.45 | +0.25 | 0 |
| IRI | $2.80 | $13.40 | +0.33 | 0 |
| SLI | $1.40 | $3.40 | +0.33 | 0.73 |

TABLE 5
Repeatabilities of EBV with different amounts of test data (from a
previous report)

| | | | | | | approx. | approx |
|---|---|---|---|---|---|---|---|
| | | | repeatability of | approximate | Approx. | average | average |
| | | | the EBV of a | repeatability | repeatability | repeatability | repeatability |
| | | | single trait | of PPD EBV | of HEWL | of | of |
| | | | with a 25% | (16% | EBV (27% | PPD | HEWL |
| sex | situation | frequency | heritability | heritability) | heritability) | EBV | EBV |
| males | dam tested | 1/3 | 42% | 27% | 45% | 26% | 44% |
| | dam | 2/3 | 40% | 26% | 43% | | |
| | untested | | | | | | |
| females | individual | 1/9 | 42% | 27% | 45% | 21% | 35% |
| | and dam | | | | | | |
| | tested | | | | | | |
| | individual | 2/9 | 40% | 26% | 43% | | |
| | tested, dam | | | | | | |
| | untested | | | | | | |
| | dam tested, | 2/9 | 30% | 19% | 32% | | |
| | individual | | | | | | |
| | untested | | | | | | |
| | individual | 4/9 | 27% | 17% | 29% | | |
| | and dam | | | | | | |
| | untested | | | | | | |

TABLE 6a
Approximate expected responses to selection among
males in a sire line (Duroc, top 10%), in phenotypic
standard deviations of each trait or index, when the
index is designed to provide equal expected response for
IR and production traits and 2/3 of females are not tested.

| | s.d. of EBV or | phenotypic | |
|---|---|---|---|
| | index | s.d. | Response |
| backfat (mm) | 1.18 | 2.12 | −0.36 |
| age (d) | 4 | 12.4 | −0.17 |
| log(PPD) | 0.0388 | 0.19 | +0.27 |
| HEWL | 0.1551 | 0.45 | +0.33 |
| IRI | $2.57 | $10.21 | +0.39 |
| SLI | $1.40 | $3.40 | +0.35 |

TABLE 6b
Approximate expected responses to selection among females
in a sire line (Duroc, top 10%), in phenotypic standard deviations
of each trait or index, when the index is designed to provide
equal expected response for IR and production traits
and 2/3 of females are not tested.

| | s.d. of EBV or | phenotypic | |
|---|---|---|---|
| | index | s.d. | Response |
| backfat (mm) | 1.18 | 2.12 | −0.40 |
| age (d) | 4 | 12.4 | −0.19 |
| log(PPD) | 0.0348 | 0.19 | +0.24 |
| HEWL | 0.1383 | 0.45 | +0.28 |
| IRI | $2.31 | $10.21 | +0.34 |
| SLI | $1.40 | $3.40 | +0.38 |

TABLE 7a
Approximate expected responses to selection among
males in a dam line (Yorkshire and Landrace, top 10%),
in phenotypic standard deviations of each trait or index,
when the index is designed to provide equal expected
response for IR and production traits and 2/3 of females
are not tested.

| | s.d. of EBV or | phenotypic | |
|---|---|---|---|
| | index | s.d. | Response |
| backfat (mm) | 1.18 | 2.12 | −0.45 |
| age (d) | 4 | 12.4 | −0.21 |
| litter size | 0.39 | 3 | +0.11 |
| log(PPD) | 0.0388 | 0.19 | +0.20 |
| HEWL | 0.1551 | 0.45 | +0.24 |
| IRI | $0.75 | $3.00 | +0.28 |
| DLI | $0.92 | $5.02 | +0.25 |

TABLE 7b
Approximate expected responses to selection among females in a
dam line (Yorkshire and Landrace, top 10%), in phenotypic standard
deviations of each trait or index, when the index is designed to provide
equal expected response for IR and production traits and 2/3 of females
are not tested.

| | s.d. of EBV or | phenotypic | |
|---|---|---|---|
| | index | s.d. | Response |
| backfat (mm) | 1.18 | 2.12 | −0.47 |
| age (d) | 4 | 12.4 | −0.22 |
| litter size | 0.39 | 3 | +0.12 |
| log(PPD) | 0.0348 | 0.19 | +0.16 |
| HEWL | 0.1383 | 0.45 | +0.20 |
| IRI | $0.68 | $3.00 | +0.24 |
| DLI | $0.92 | $5.02 | +0.26 |

TABLE 8
Approximate expected annual responses to selection, in
phenotypic standard deviations of each trait or index, when the index
is designed to provide equal expected response for IR and production
traits, 2/3 of females are not tested, 11% of males and 26% of females
are selected, and generation intervals are 12 and 18 months in males
and females respectively.

| | | |
|---|---|---|
| s.d. of EBV or | phenotypic | Annual |
| index | s.d. | response |

a) Dam line

| | | | |
|---|---|---|---|
| backfat (mm) | 1.18 | 2.12 | −0.31 |
| age (d) | 4 | 12.4 | −0.14 |
| litter size | 0.39 | 3 | +0.08 |
| log(PPD) | 0.0388 in males, | 0.19 | +0.12 |
| | 0.0348 in | | |
| | females | | |
| HEWL | 0.1551 in males, | 0.45 | +0.15 |
| | 0.1383 in | | |
| | females | | |
| IRI | $0.75 in males, | $3.00 | +0.17 |
| | $0.68 in females | | |
| DLI | $1.40 | $3.40 | +0.17 |

b) Sire line

| | | | |
|---|---|---|---|
| backfat (mm) | 1.18 | 2.12 | −0.25 |
| age (d) | 4 | 12.4 | −0.12 |
| log(PPD) | 0.0388 in males, | 0.19 | +0.17 |
| | 0.0348 in | | |
| | females | | |
| HEWL | 0.1551 in males, | 0.45 | +0.21 |
| | 0.1383 in | | |
| | females | | |
| IRI | $2.57 in males, | $10.21 | +0.25 |
| | $2.31 in females | | |
| SLI | $1.40 | $3.40 | +0.25 |

TABLE 9a
Approximate expected responses to selection among males in a
sire line (Duroc, top 10%), in phenotypic standard deviations of each
trait or index, when the index gives equal economic value to one
phenotypic standard deviation of IRI and one phenotypic standard
deviation of SLI and 2/3 of females are not tested.

| | s.d. of EBV or | phenotypic | |
|---|---|---|---|
| | index | s.d. | Response |
| backfat (mm) | 1.18 | 2.12 | −0.65 |
| age (d) | 4 | 12.4 | −0.30 |
| log(PPD) | 0.0388 | 0.19 | +0.16 |
| HEWL | 0.1551 | 0.45 | +0.19 |
| IRI | $0.85 | $3.38 | +0.23 |
| SLI | $1.40 | $3.38 | +0.62 |

TABLE 9b
Approximate expected responses to selection among females in a
sire line (Duroc, top 10%), in phenotypic standard deviations of each
trait or index, when the index gives equal economic value to one
phenotypic standard deviation of IRI and one phenotypic standard
deviation of SLI and 2/3 of females are not tested.

| | s.d. of EBV or | phenotypic | |
|---|---|---|---|
| | index | s.d. | Response |
| backfat (mm) | 1.18 | 2.12 | −0.67 |
| age (d) | 4 | 12.4 | −0.31 |
| log(PPD) | 0.0348 | 0.19 | +0.13 |
| HEWL | 0.1383 | 0.45 | +0.16 |
| IRI | $0.76 | $3.38 | +0.19 |
| SLI | $1.40 | $3.38 | +0.64 |

TABLE 10a
Approximate expected responses to selection among males in a
dam line (Yorkshire and Landrace, top 10%), in phenotypic standard
deviations of each trait or index, when the index gives equal economic
value to one phenotypic standard deviation of IRI and one phenotypic
standard deviation of DLI and 2/3 of females are not tested.

| | s.d. of EBV or | phenotypic | |
|---|---|---|---|
| | index | s.d. | Response |
| backfat (mm) | 1.18 | 2.12 | −0.34 |
| age (d) | 4 | 12.4 | −0.16 |
| litter size | 0.39 | 3 | +0.09 |
| log(PPD) | 0.0388 | 0.19 | +0.25 |
| HEWL | 0.1551 | 0.45 | +0.30 |
| IRI | $1.25 | $5.02 | +0.35 |
| DLI | $0.92 | $5.02 | +0.19 |

TABLE 10b
Approximate expected responses to selection among females in
a dam line (Yorkshire and Landrace, top 10%), in phenotypic standard
deviations of each trait or index, when the index gives equal economic
value to one phenotypic standard deviation of IRI and one phenotypic
standard deviation of DLI and 2/3 of females are not tested.

| | s.d. of EBV or | phenotypic | |
|---|---|---|---|
| | index | s.d. | Response |
| backfat (mm) | 1.18 | 2.12 | −0.37 |
| age (d) | 4 | 12.4 | −0.17 |
| litter size | 0.39 | 3 | +0.09 |
| log(PPD) | 0.0348 | 0.19 | +0.21 |
| HEWL | 0.1383 | 0.45 | +0.26 |
| IRI | $1.12 | $5.02 | +0.30 |
| DLI | $0.92 | $5.02 | +0.20 |

TABLE 11
Approximate expected annual responses to selection, in
phenotypic standard deviations of each trait or index, when the
index gives equal economic value to one phenotypic standard
deviation of IRI and one phenotypic standard deviation of DLI,
2/3 of females are not tested, 11% of males and 26% of females
are selected, and generation intervals are 12 and 18 months in
males and females respectively.

| | | |
|---|---|---|
| s.d. of EBV or | phenotypic | Annual |
| index | s.d. | response |

a) Dam line

| | | | |
|---|---|---|---|
| backfat (mm) | 1.18 | 2.12 | −0.24 |
| age (d) | 4 | 12.4 | −0.11 |
| litter size | 0.39 | 3 | +0.06 |
| log(PPD) | 0.0388 in males, | 0.19 | +0.16 |
| | 0.0348 in | | |
| | females | | |
| HEWL | 0.1551 in males, | 0.45 | +0.19 |
| | 0.1383 in | | |
| | females | | |
| IRI | $1.25 in males, | $5.02 | +0.22 |
| | $1.12 in females | | |
| DLI | $1.40 | $5.02 | +0.13 |

b) Sire line

| | | | |
|---|---|---|---|
| backfat (mm) | 1.18 | 2.12 | −0.44 |
| age (d) | 4 | 12.4 | −0.20 |
| log(PPD) | 0.0388 in males, | 0.19 | +0.10 |
| | 0.0348 in | | |
| | females | | |
| HEWL | 0.1551 in males, | 0.45 | +0.12 |
| | 0.1383 in | | |
| | females | | |
| IRI | $0.85 in males, | $3.38 | +0.14 |
| | $0.76 in females | | |
| SLI | $1.40 | $3.38 | +0.42 |

TABLE 12
Expected proportions of pigs above the original unselected mean
phenotypic IRI after 0 to 5 years of selection on SI.
a) Dam lines. Equal response SI($) = 1.54LITTER SIZE −
0.45FAT − 0.11AGE + 14.10PPD + 3.02HEWL
(plotted with legend DL-ER)

| | expected mean IRI (phenotypic | proportion above original |
|---|---|---|
| Year | s.d. of improvement) | phenotypic mean |
| 0 | 0 | 50% |
| 1 | 0.17 | 57% |
| 2 | 0.34 | 63% |
| 3 | 0.51 | 70% |
| 4 | 0.68 | 75% |
| 5 | 0.85 | 80% |

b) Dam lines. Equal value SI($) = 1.54LITTER SIZE − 0.45FAT −
0.11AGE + 23.28PPD + 4.99HEWL
(plotted with legend DL-EV)

| | mean IRI (phenotypic s.d. of | proportion above original |
|---|---|---|
| Year | improvement) | phenotypic mean |
| 0 | 0 | 50% |
| 1 | 0.22 | 59% |
| 2 | 0.44 | 67% |
| 3 | 0.66 | 75% |
| 4 | 0.88 | 81% |
| 5 | 1.10 | 86% |

c) Sire lines. Equal response SI($) = −0.92FAT − 0.22AGE +
47.94PPD + 10.27HEWL
(plotted with legend SL-ER)

| | mean IRI (phenotypic s.d. of | proportion above original |
|---|---|---|
| Year | improvement) | phenotypic mean |
| 0 | 0 | 50% |
| 1 | 0.25 | 60% |
| 2 | 0.50 | 69% |
| 3 | 0.75 | 77% |
| 4 | 1.00 | 84% |
| 5 | 1.25 | 89% |

d) Sire lines. Equal value SI($) = −0.92FAT − 0.22AGE +
15.77PPD + 3.38HEWL
(plotted with legend SL-EV)

| | mean IRI (phenotypic s.d. of | proportion above original |
|---|---|---|
| Year | improvement) | phenotypic mean |
| 0 | 0 | 50% |
| 1 | 0.14 | 56% |
| 2 | 0.28 | 61% |
| 3 | 0.42 | 66% |
| 4 | 0.56 | 71% |
| 5 | 0.70 | 76% |

### Example 6

### Data generated on pigs during selection of high immune response

Table 13 shows an example of data generated from pigs selected for immune response and performance testing in a commercial breeding herd of Yorkshire, Landrace and Duroc pigs during the week of Apr. 30, 2001. The phenotypic value of each pig for cell mediated immune response and the EBV for that trait are shown in the two columns labelled PPD. The phenotypic value of each pig for antibody response on days 0 to 21 are shown in the columns labelled Day 0-21, respectively. The EBV for antibody response is shown in the column labelled HEWL. The immune response index for each pig is shown in the column labelled IR. The production index for each pigs is shown in the column labelled PI and the selection index, which is a reflection of both immune response and production EBVs, is shown in the column labelled SI. Other information on the pig, such as tag number, tattoo number, barn location, and accuracy of the EBVs are also given in the table. In this example, the information is separated on the bases of breed (DU=Duroc, LA=Landrace, YO=Yorkshire) and sex (M=male, F=female) of pig, but the data is not ordered according to IR, PI or SI, although it is possible to order the data and rank pigs according to any of these variables and use this information as a bases to breed pigs for HIR.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims

All publications, patents and patent applications are herein incorporated by reference in their entirety to the same extent as if each individual publication, patent or patent application was specifically and individually indicated to be incorporated by reference in its entirety.

TABLE 13
Apr. 20, 2001 Weeks 3 to 3
Ordered By Sex, Breed, Day 21 “HEWL Desc”

| | | | | | Cull | | EBV | | | | | | | | | |
|---|---|---|---|---|---|---|---|---|---|---|---|---|---|---|---|---|
| Tag | Tattoo | Sex | Birth | Tst | St | Osp | Fat | Age | Ind T | Brn | Rpt | DLI | Rpt | PPD | PPO | Rpt |

DUDUDUDU

| 1813 | ANZD 5612K | M | 00/12/18 | T | | | −0.2 | 2.6 | 92 | 0.306 | 10 | 108 | 18 | 107.61 | 0.0690 | 22 |
|---|---|---|---|---|---|---|---|---|---|---|---|---|---|---|---|---|
| 1797 | ANZD 3091K | M | 00/12/15 | T | C S | | 0.8 | 0.2 | 85 | −0.047 | 8 | 88 | 16 | 13.85 | −0.0671 | 25 |
| 1811 | ANZD 5601K | M | 00/12/18 | T | | | −0.3 | −1.3 | 111 | 0.009 | 11 | 108 | 17 | 78.78 | 0.0330 | 21 |
| 1798 | ANZD 3092K | M | 00/12/15 | T | | | 0.8 | 0.2 | 86 | −0.047 | 8 | 88 | 16 | 49.07 | −0.0305 | 25 |
| 1801 | ANZD 3131K | M | 00/12/14 | T | | | 0.7 | −0.8 | 92 | −0.069 | 14 | 91 | 19 | 81.17 | 0.0498 | 26 |
| 1812 | ANZD 5602K | M | 00/12/18 | T | | | −0.3 | −1.3 | 111 | 0.009 | 11 | 108 | 17 | 66.06 | 0.0231 | 21 |
| 1800 | ANZD 3121K | M | 00/12/15 | T | | | 0.5 | −0.8 | 95 | 0.520 | 14 | 117 | 19 | 51.54 | 0.0199 | 26 |
| 1802 | ANZD 3122K | M | 00/12/15 | T | | | 0.5 | −0.8 | 95 | 0.520 | 14 | 117 | 19 | 69.66 | 0.0352 | 26 |
| 1803 | ANZD 3151K | M | 00/12/17 | T | C S | | 1.6 | −0.8 | 77 | 0.765 | 14 | 114 | 19 | 63.63 | 0.0301 | 26 |
| 1799 | ANZD 3111K | M | 00/12/15 | T | C S | | 1.3 | −1.1 | 83 | −0.060 | 15 | 86 | 20 | 1.02 | −0.0343 | 26 |
| 1796 | ANZD 3081K | M | 00/12/15 | T | C S | | 0.1 | 1.8 | 91 | −0.254 | 13 | 84 | 20 | 40.16 | −0.0220 | 24 |
| | ANZD 3141K | M | 00/12/15 | T | | X | −0.5 | 0.9 | 105 | −0.283 | 9 | 92 | 15 | 0.00 | 0.0204 | 3 |
| Total DUDUDUDU Listed | | | | 12 | | | | | | | | | | | | |
| 1829 | ANZD 3132K | F | 00/12/14 | T | | | 0.7 | −0.8 | 92 | −0.069 | 14 | 91 | 19 | 133.74 | 0.0904 | 26 |
| 1822 | ANZD 3143K | F | 00/12/16 | T | | | −0.5 | 0.9 | 105 | −0.283 | 9 | 92 | 15 | 82.26 | 0.0387 | 19 |
| 1827 | ANZD 3113K | F | 00/12/15 | T | | | 1.3 | −1.1 | 83 | −0.060 | 15 | 86 | 20 | −14.25 | −0.0500 | 26 |
| 1844 | ANZD 3133K | F | 00/12/14 | T | | | 0.7 | −0.8 | 92 | −0.069 | 14 | 91 | 19 | 54.72 | 0.0350 | 26 |
| 1828 | ANZD 3123K | F | 00/12/15 | T | | | 0.5 | −0.8 | 95 | 0.520 | 14 | 117 | 19 | 67.68 | 0.0399 | 26 |
| 1841 | ANZD 3083K | F | 00/12/15 | T | | | 0.1 | 1.8 | 91 | −0.254 | 13 | 84 | 20 | 49.89 | −0.0065 | 24 |
| 1830 | ANZD 3153K | F | 00/12/17 | T | | | 1.6 | −0.8 | 77 | 0.765 | 14 | 114 | 19 | 29.51 | 0.0048 | 26 |
| 1831 | ANZD 5603K | F | 00/12/18 | T | | | −0.3 | −1.3 | 111 | 0.009 | 11 | 108 | 17 | 72.83 | 0.0347 | 21 |
| 1843 | ANZD 3094K | F | 00/12/15 | T | | | 0.8 | 0.2 | 86 | −0.047 | 8 | 88 | 16 | 24.49 | −0.0486 | 25 |
| 1832 | ANZD 5614K | F | 00/12/18 | T | | | −0.2 | 2.6 | 92 | 0.306 | 10 | 108 | 18 | 81.69 | 0.0575 | 22 |
| 1826 | ANZD 3112K | F | 00/12/15 | T | | | 1.3 | −1.1 | 83 | −0.060 | 15 | 86 | 20 | 65.76 | 0.0379 | 26 |
| 1842 | ANZD 3093K | F | 00/12/15 | T | | X | 0.8 | 0.2 | 86 | −0.047 | 8 | 88 | 16 | 0.00 | −0.0389 | 10 |
| Total DUDUDUDU Listed | | | | 12 | | | | | | | | | | | | |
| LALALALA | | | | | | | | | | | | | | | | |
| 1805 | ASDZ 1881K | M | 00/12/16 | T | | | 0.0 | 1.1 | 96 | −0.210 | 16 | 90 | 21 | 86.79 | 0.0383 | 22 |
| 1809 | ASDZ 5771K | M | 00/12/18 | T | | | 0.6 | 4.0 | 77 | 0.214 | 16 | 91 | 22 | 36.32 | −0.0256 | 21 |
| 1807 | ASDZ 5731K | M | 00/12/15 | T | | | 0.8 | 4.7 | 71 | 0.602 | 12 | 101 | 19 | 67.32 | 0.0089 | 21 |
| 1810 | ASDZ 5781K | M | 00/12/18 | T | C S | | 1.4 | 0.3 | 78 | 0.082 | 12 | 88 | 15 | 21.67 | −0.0493 | 21 |
| 1806 | ASDZ 5721K | M | 00/12/13 | T | | | 0.2 | 1.2 | 93 | 0.305 | 5 | 105 | 12 | 55.68 | −0.0106 | 23 |

| | | | | | | | | | | | | | | | |
|---|---|---|---|---|---|---|---|---|---|---|---|---|---|---|---|
| Tag | Tattoo | Sex | Birth | Day 0 | Day 14 | Day 21 | HEWL | Rpt | IR Ind | Rp | Clc | SI | Rpt | PI | Rpt |

DUDUDUDU

| | | | | | | | | | | | | | | | |
|---|---|---|---|---|---|---|---|---|---|---|---|---|---|---|---|
| 1813 | ANZD 5612K | M | 00/12/18 | 0.0872 | 0.0510 | 0.9571 | 0.1267 | 30 | 12.93 | 26 | H | 3.90 | 38 | −0.39 | 0 |
| 1797 | ANZD 3091K | M | 00/12/15 | 0.2414 | 0.1548 | 0.9168 | 0.2049 | 33 | 0.29 | 29 | H | −0.68 | 41 | −0.78 | 0 |
| 1811 | ANZD 5601K | M | 00/12/18 | 0.0365 | 0.0408 | 0.8677 | 0.0945 | 30 | 7.51 | 25 | H | 3.06 | 36 | 0.56 | 0 |
| 1798 | ANZD 3092K | M | 00/12/15 | 0.1459 | 0.1023 | 0.5983 | 0.1334 | 33 | 1.71 | 29 | H | −0.21 | 41 | −0.78 | 0 |
| 1801 | ANZD 3131K | M | 00/12/14 | 0.1130 | 0.1687 | 0.5017 | 0.0049 | 34 | 5.94 | 30 | H | 1.50 | 42 | −0.47 | 0 |
| 1812 | ANZD 5602K | M | 00/12/18 | 0.0529 | 0.1013 | 0.4974 | 0.0125 | 30 | 3.16 | 25 | H | 1.61 | 36 | 0.56 | 0 |
| 1800 | ANZD 3121K | M | 00/12/15 | 0.0841 | 0.0854 | 0.3462 | −0.0382 | 34 | 0.80 | 30 | H | −0.02 | 42 | −0.28 | 0 |
| 1802 | ANZD 3122K | M | 00/12/15 | 0.1426 | 0.1275 | 0.2322 | −0.0636 | 34 | 1.57 | 30 | H | 0.24 | 42 | −0.28 | 0 |
| 1803 | ANZD 3151K | M | 00/12/17 | 0.1246 | 0.1342 | 0.2310 | −0.0216 | 34 | 2.63 | 30 | H | −0.42 | 42 | −1.30 | 0 |
| 1799 | ANZD 3111K | M | 00/12/15 | 0.1100 | 0.1554 | 0.1448 | −0.0844 | 34 | −7.27 | 30 | H | −3.37 | 42 | −0.95 | 0 |
| 1796 | ANZD 3081K | M | 00/12/15 | 0.1064 | 0.0900 | 0.1027 | −0.0681 | 32 | −5.21 | 28 | H | −2.22 | 40 | −0.49 | 0 |
| | ANZD 3141K | M | 00/12/15 | 0.0000 | 0.0000 | 0.0000 | 0.0675 | 4 | 5.00 | 3 | S | 1.92 | 9 | 0.26 | 0 |

Total DUDUDUDU Listed

| | | | | | | | | | | | | | | | |
|---|---|---|---|---|---|---|---|---|---|---|---|---|---|---|---|
| 1829 | ANZD 3132K | F | 00/12/14 | 0.0895 | 0.1035 | 0.7453 | 0.0850 | 34 | 13.76 | 30 | H | 4.10 | 42 | −0.47 | 0 |
| 1822 | ANZD 3143K | F | 00/12/16 | 0.2343 | 0.1035 | 0.7219 | 0.1181 | 27 | 9.10 | 23 | H | 3.28 | 33 | 0.26 | 0 |
| 1827 | ANZD 3113K | F | 00/12/15 | 0.0515 | 0.2508 | 0.4379 | 0.0060 | 34 | −5.53 | 30 | H | −2.79 | 42 | −0.95 | 0 |
| 1844 | ANZD 3133K | F | 00/12/14 | 0.0977 | 0.1832 | 0.3645 | 0.0007 | 34 | 4.07 | 30 | H | 0.88 | 42 | −0.47 | 0 |
| 1828 | ANZD 3123K | F | 00/12/15 | 0.0913 | 0.2011 | 0.2390 | −0.0356 | 34 | 3.21 | 30 | H | 0.78 | 42 | −0.28 | 0 |
| 1841 | ANZD 3083K | F | 00/12/15 | 0.1071 | 0.0842 | 0.2040 | −0.0189 | 32 | −1.49 | 28 | H | −0.98 | 40 | −0.49 | 0 |
| 1830 | ANZD 3153K | F | 00/12/17 | 0.1494 | 0.1273 | 0.1951 | −0.0032 | 34 | 0.43 | 30 | H | −1.15 | 42 | −1.30 | 0 |
| 1831 | ANZD 5603K | F | 00/12/18 | 0.0590 | 0.0572 | 0.1014 | −0.0487 | 30 | 2.10 | 25 | H | 1.26 | 36 | 0.56 | 0 |
| 1843 | ANZD 3094K | F | 00/12/15 | 0.1273 | 0.0867 | 0.0860 | 0.0454 | 33 | −3.83 | 29 | H | −2.05 | 41 | −0.78 | 0 |
| 1832 | ANZD 5614K | F | 00/12/18 | 0.0793 | 0.0747 | 0.0714 | −0.0400 | 30 | 5.07 | 26 | H | 1.29 | 38 | −0.39 | 0 |
| 1826 | ANZD 3112K | F | 00/12/15 | 0.0273 | 0.0302 | 0.0528 | −0.0784 | 34 | 1.30 | 30 | H | −0.52 | 42 | −0.95 | 0 |
| 1842 | ANZD 3093K | F | 00/12/15 | 0.0000 | 0.0000 | 0.0000 | 0.1273 | 12 | 0.50 | 11 | S | −0.61 | 19 | −0.78 | 0 |

Total DUDUDUDU Listed
LALALALA

| | | | | | | | | | | | | | | | |
|---|---|---|---|---|---|---|---|---|---|---|---|---|---|---|---|
| 1805 | ASDZ 1881K | M | 00/12/16 | 0.0500 | 0.8171 | 1.4227 | 0.0498 | 30 | 6.37 | 26 | H | 0.18 | 22 | −0.44 | 0 |
| 1809 | ASDZ 5771K | M | 00/12/18 | 0.0823 | 0.1961 | 1.4096 | 0.0393 | 29 | −1.41 | 25 | H | −0.53 | 23 | −0.39 | 0 |
| 1807 | ASDZ 5731K | M | 00/12/15 | 0.0984 | 0.1381 | 1.1728 | −0.0253 | 30 | 0.04 | 25 | H | 0.05 | 21 | 0.04 | 0 |
| 1810 | ASDZ 5781K | M | 00/12/18 | 0.0779 | 0.4840 | 1.1645 | −0.0140 | 29 | −6.24 | 25 | H | −1.16 | 18 | −0.55 | 0 |
| 1806 | ASDZ 5721K | M | 00/12/13 | 0.0939 | 0.3168 | 1.1137 | −0.0601 | 31 | −3.58 | 27 | H | −0.11 | 17 | 0.25 | 0 |

Full Citations for References Referred to in the Specification

Brown, W. C. et al., (1998) “Bovine Type 1 and Type 2 Responses”, Vet. Immunl. Immunopath 63:45-55.

Nicholas, F. W. Veterinary Genetics, Oxford Science Publications, 1987.

Falconer, D. S. (1981) “An introduction to quantitative genetics.” Longman, London.

Molitor, T. and L. Schwandtdt, “Role of Stress on Mediating Disease in Animals”, Proc. Stress Symposia: Mechanisms, Responses, Management. Ed., N. H. Granholm, South Dakota State University Press, Apr. 6-7, 1993.

Morrow-Tesch J. L. et al. (1996), J. Therm. Biol. 21(2):101-108.

Johnson, L. G. “Temperature Tolerance, Temperature Stress, and Animal Development” Proc. Stress Symposia: Mechanisms, Responses, Management. Ed., N. H. Granholm, South Dakota State University Press, Apr. 6-7, 1998.

McGloner, J. J. “Indicators of Stress in Livestock and Implications for Advancements in Livestock Housing”, Proc. Stress Symposia: Mechanisms, Responses, Management. Ed., N. H. Granhoim, South Dakota State University Press, Apr. 6-7, 1998.

Hessing M. J. C. et al. (1994) “Social Rank and Disease Susceptibility in Pigs”, Vet. Immunol. Immunopath 43:373-387.

Blecha, F. (1985) “Immunoligcal Reactions of Pigs Regrouped at or Near Weaning”, Am J. Vet. Res. 46(9):1934-1937.

Thompson et al. (1980) “Cell Mediated Immunity in Marek's Disease Virus-Infected Chickens Genetically Selected for High and Low Concentrations of Plasma Corticosterone”, Am. J. Vet. Res. 41(1):91-96.

Kehrli, H. E. et al. 1989 a & b, Am J. Vet. Res. 50(2):207 and 215.

1. A method for predicting an animal's level of immune response, disease resistance or susceptibility, and/or productivity based on an Estimated Breeding Value (EBV) of the animal's immune responsiveness, comprising:
(i) determining a heritable antibody response of a test animal by measuring, in the test animal, the levels of antibody which are specifically induced to a predetermined antigen;
(ii) determining a cell-mediated immune response trait of the test animal by measuring, in the test animal, a cell-mediated immune response which is specifically induced to a predetermined antigen;
(iii) calculating an EBV for the test animal which is based on the determinations in (i) and (ii); and
(iv) comparing the test animal's EBV to EBVs obtained for other animals within the population of animals, and thereby assigning the test animal to a high, low, or control EBV group, wherein a high, low, or control EBV correlates with a predicted level of immune response, disease resistance or susceptibility, and/or productivity in the animal.

2. A method for obtaining a group of animals which has a predicted level of immune response, disease resistance or susceptibility, and/or a group of animals which has a predicted productivity which comprises:
(i) determining a heritable antibody response trait of a test animal by measuring in the test animal the levels of antibody which are specifically induced to a predetermined antigen;
(ii) determining a cell-mediated immune response trait of the test animal by measuring in the test animal a cell-mediated immune response which is specifically induced to a predetermined antigen;
(iii) calculating an EBV for the test animal based on the determinations in (i) and (ii); and
(iv) comparing the test animal's EBV to EBVs obtained for other animals within a population of animals and thereby assigning the test animal to a high, low or control EBV group; and
(v) selecting animals in one of the high, low or control EBV groups and breeding the animals to produce a group of animals which have a predicted level of immune response, disease resistance or susceptibility, and/or a group of animals which has a predicted productivity.

3. A method of determining the efficacy of a vaccine, drug or other treatment in an animal comprising:
(i) determining a heritable antibody response trait of a test animal by measuring in the test animal the levels of antibody which are specifically induced to a predetermined antigen;
(ii) determining a cell-mediated immune response trait of the test animal by measuring in the test animal a cell-mediated immune response which is specifically induced to a predetermined antigen;
(iii) calculating an EBV for the test animal based on the determinations in (i) and (ii);
(iv) comparing the test animal's EBV to EBVs obtained for other animals within a population of animals and thereby assigning the test animal to a high, low or control EBV group; and
(v) administering the vaccine, drug, or other treatment to animals in one or more of the high, low or control EBV groups, and comparing the responses to the vaccine, drug or other treatment in one or more of the high, low and control EBV groups, wherein a positive response to the vaccine, drug or other treatment in the high EBV group only, indicates that the vaccine, drug or other treatment has low efficacy, and wherein a positive response to the vaccine, drug or other treatment in the high, control and low EBV groups indicates that the vaccine, drug or other treatment has high efficacy.

4. The method of claim 1 wherein the animals are selected from the group consisting of: swine, cattle, horses, poultry, fish, cats and dogs.

5. The method of claim 1 wherein the animals are swine.

6. The method of claim 1 wherein the predetermined antigen which specifically induces an antibody response and the predetermined antigen which specifically induces a cell mediated immune response are different antigens.

7. The method of claim 6 wherein the antigens are selected from a group of antigens to which the animals are not normally exposed.

8. The method of claim 7 wherein the antigens are selected from a group of antigens to which the animals have not been previously exposed.

9. The method of claim 8 wherein the antigens are selected from the group of antigens consisting of: Hen Egg White Lysozyme (HEWL), ovalbumin, human serum albumin, and tyrosine-glycine-alanine copolymer ((TG)-A-L).

10. The method of claim 1, wherein,
(I) the heritable antibody response trait of a test animal is determined by:
(a) immunizing the test animal at least once with at least one antigen which can evoke a specific antibody response;
(b) for the test animal, measuring a specific antibody response to the at least one antigen at least once; and
(II) the cell mediated immune response trait of the animal is determined by:
(c) exposing the test animal to an antigen which can evoke a specific cell-mediated immune response (CMIR); and
(d) measuring at least one indicator of the CMIR of the test animal.

11. The method of claim 10, wherein the test animal is immunized at least two times with at least one antigen which can evoke a specific antibody response and is exposed at least two times to an antigen which can evoke a specific CMIR.

12. A method of claim 10 wherein the indicator of the CMIR is selected from the group consisting of cytokines; delayed type hypersensitivity, and in vitro lymphocyte proliferation to at least one antigen.

13. The method of claim 10 wherein the animals are immunized at a time when they are most at risk for disease.

14. The method of claim 13 wherein the animals are swine and are immunized after weaning at an age of about 21 days.

15. A use of the method of claim 1 selected from the group of uses consisting of: determining the efficacy of a drug or vaccine in an animal; obtaining a group of animals which has a predicted level of immune response, disease resistance or susceptibility, and/or a group of animals which has a predicted productivity; selecting an animal having a predicted level of immune response, disease resistance or susceptibility, and/or productivity; selecting an animal having predicted stress coping abilities; and obtaining a group of animals having predicted stress coping abilities, based on an EBV of the animal's immune responsiveness.

16. A method of predicting the level of immune response, disease resistance or susceptibility, and/or productivity of a test animal within a population of animals based on an EBV of the animal's immune responsiveness comprising:
(a) immunizing the test animal at least once with at least one antigen which can evoke a specific antibody response;
(b) for the test animal, measuring a specific antibody response to the at least one antigen at least once;
(c) exposing the test animal to an antigen which can evoke a specific cell-mediated immune response (CMIR); and
(d) measuring at least one indicator of the CMIR of the test animal,
(e) calculating the EBV for the test animal based on the determinations in (b) and (d); and
(f) comparing the test animal's EBV to EBVs obtained for the other animals within the population of animals and thereby assigning the test animal to a high, low, or control EBV group, wherein a high, low or control EBV correlates with a predicted level of immune response, disease resistance or susceptibility, and/or productivity in the test animal.

17. The method of claim 1 wherein the antibody response and the cell-mediated immune response traits of the animal are determined when the animal is under stress.

18. The use of the method of claim 17 for selecting an animal having predicted stress coping abilities based on the EBV of the animal's immune responsiveness under stress.

19. The use of the method of claim 1, for predicting an animal's level of growth hormones.

20. The use of the method of claim 2, for obtaining a group of animals having a predicted level of growth hormones.

21. A method for selecting an animal having a predicted level of immune response, disease resistance or susceptibility, and/or productivity based on the EBV of at least one of the animal's relatives as determined in accordance with the method of claim 1.
